(19) United States
(12) Patent Application Publication
(10) Pub. No.: US 2019/0127772 A1
(43) Pub. Date: May 2, 2019

(54) ENZYMATIC GLYCOSYLATION OF STEVIOL GLYCOSIDES AND OTHER COMPOUNDS WITH GLUCOSE-1-PHOSPHATE

(71) Applicant: Codexis, Inc., Redwood City, CA (US)

(72) Inventors: Jonathan Vroom, South San Francisco, CA (US); Stephanie Sue Galanie, Knoxville, TN (US); Oscar Alvizo, Fremont, CA (US)

(21) Appl. No.: 16/306,015

(22) PCT Filed: Jun. 9, 2017
(86) PCT No.: PCT/US17/36701

§ 371 Date: Nov. 30, 2018

Related U.S. Application Data
(60) Provisional application No. 62/350,450, filed on Jun. 15, 2016.

(51) Int. Cl. C12P 19/18; C12P 19/56; C12P 19/02

(52) U.S. Cl. CPC C12P 19/18; C12P 19/02; C12P 19/56

(57) ABSTRACT

The present invention provides glycosyl transferase (GT) enzymes, polypeptides having GT activity, and polynucleotides encoding these enzymes, as well as vectors and host cells comprising these polynucleotides and polypeptides. The present invention also provides methods of using these GT enzymes to generate products with β-glucose linkages.

The present application claims priority to U.S. Prov. Pat. Appln. Ser. No. 62/350,450, filed Jun. 15, 2016, hereby incorporated by reference in its entirety for all purposes.

### FIELD OF THE INVENTION

The present invention provides glycosyltransferase (GT) enzymes, polypeptides having GT activity, and polynucleotides encoding these enzymes, as well as vectors and host cells comprising these polynucleotides and polypeptides. The present invention also provides methods of using these GT enzymes to generate products with β-glucose linkages.

### REFERENCE TO A “SEQUENCE LISTING,” A TABLE, OR A COMPUTER PROGRAM LISTING APPENDIX SUBMITTED AS AN ASCII TEXT FILE

The official copy of the Sequence Listing is submitted concurrently with the specification as an ASCII formatted text file via EFS-Web, with a file name of “CX8-155USP1_ST25.txt”, a creation date of Jun. 14, 2016, and a size of 98,304 bytes. The Sequence Listing filed via EFS-Web is part of the specification and is incorporated in its entirety by reference herein.

### BACKGROUND OF THE INVENTION

Glycosyltransferases (GT) are enzymes that post-translationally transfer glycosyl residues from an activated nucleotide sugar to monomeric and polymeric acceptor molecules (e.g., other sugars, proteins, lipids, and other organic substrates). These glycosylated molecules are involved in various metabolic pathways and processes. The transfer of a glucosyl moiety can alter the acceptor's bioactivity, solubility, and transport properties within cells.

### SUMMARY OF THE INVENTION

The present invention provides glycosyltransferase enzymes, polypeptides having GT activity, and polynucleotides encoding these enzymes, as well as vectors and host cells comprising these polynucleotides and polypeptides. The present invention also provides methods of using these GT enzymes to generate products with β-glucose linkages.

The present invention provides methods for glycosylation of a substrate to produce a beta-glycosylated product, comprising the steps of providing at least one glycosyl group donor, a least one glycosyl group acceptor, and at least one glycosyltransferase enzyme; contacting the glycosyl group donor and glvcosyl group acceptor with the glycosyltransferase enzyme under conditions such that the glycosyl group acceptor is glycosylated to produce at least one product having beta-glucose linkages. In some embodiments of the methods, the glycosyl group donor is a glycosylphosphate. In some additional embodiments of the methods, the glycosyl group donor is glucose-1-phosphate. In some further embodiments of the methods, the glycosyl group acceptor is selected from glycosyl, alkoxy, carboxy, aminocarbonyl, heteroalkyl, heteroalkenyl, heteroalkynyl, carboxyalkyl, aminoalkyl, haloalkyl, alkylthioalkyl, heterocycloalkyl, heteroaryl, and heteroarylalkyl groups. In some yet additional embodiments of the methods, the product having beta-glucose linkages is a steviol glycoside. In some further embodiments of the methods, the glycosyl group acceptor is stevioside, said glycosyl group donor is alpha-glucose-1-phosphate, and said product having beta-glucose linkages is rebaudioside A. In some additional embodiments of the methods, the glycosyltransferase is selected from the polypeptides set forth in SEQ ID NOS:2, 4, 6, 8, 10, 12, and 14.

The present invention also provides methods for production of glucose-1-phosphate, comprising the steps of: providing a phosphorylase, inorganic phosphate, and a disaccharide, trisaccharide, or oligosaccharide substrate of the phosphorylase; contacting said phosphorylase, inorganic phosphate, and saccharide under conditions such that said saccharide is cleaved to produce a monosaccharide and glucose-1-phosphate. In some embodiments of the methods, this method is combined with the previously described method. In some additional embodiments of the methods, the glucosyl group acceptor is selected from glycosyl, alkoxy, carboxys, aminocarbonyl, heteroalkyl, heteroalkenyl, heteroalkynyl, carboxyalkyl, aminoalkyl, haloalkyl, alkylthioalkyl, heterocycloalkyl, heteroaryl, and heteroarylalkyl groups. In still some additional embodiments of the methods, the phosphorylase is sucrose phosphorylase, said saccharide is sucrose, said monosaccharide produced is sucrose, and said glucose-1-phosphate produced is α-glucose-1-phosphate. In some further embodiments of the methods, the phosphorylase comprises a polypeptide sequence selected from SEQ ID NOS:16, 18, 20, 22, 24, 26, 28, 30, and 32.

### DESCRIPTION OF THE DRAWINGS

FIG. 1 provides an enzymatic reaction scheme in which a glycosyltransferase catalyzes the transfer of a glycosyl group from a glycosylphosphate, for example glucose-1-phosphate, to an acceptor, for example R—OH, where R is any glycosyl, alkoxy, carboxy, aminocarbonyl, heteroalkyl, heteroalkenyl, heteroalkynyl, carboxyalkyl, aminoalkyl, haloalkyl, alkylthioalkyl, heterocycloalkyl, heteroaryl, or heteroarylalkyl group.

FIG. 2 provides a schematic of an embodiment of the invention in which the enzymatic reaction described in FIG. 1 is applied to the substrate stevioside and the glycosylphosphate is α-glucose-1-phosphate and catalyzes the formation of a β-glucose linkage, to produce the product rebaudioside A.

FIG. 3 provides a schematic of an embodiment of the invention in which two enzymatic reactions are paired for in situ generation of glucose-1-phosphate. In one reaction, sucrose phosphorylase uses inorganic phosphate to cleave sucrose, affording α-glucose-1-phosphate and fructose, and in the other reaction the glycosyl group from α-glucose-1-phosphate is transferred to an acceptor. R is any glycosyl, alkoxy, carboxy, aminocarbonyl, heteroalkyl, heteroalkenyl, heteroalkynyl, carboxyalkyl, aminoalkyl, haloalkyl, alkylthioalkyl, heterocycloalkyl, heteroaryl, or heteroarylalkyl group.

### DESCRIPTION OF THE INVENTION

The present invention provides glycosyltransferase (GT) enzymes, polypeptides having GT activity, and polynucleotides encoding these enzymes, as well as vectors and host cells comprising these polynucleotides and polypeptides. The present invention also provides methods of using these GT enzymes to generate products with β-glucose linkages.

Unless defined otherwise, all technical and scientific terms used herein generally have the same meaning as commonly understood by one of ordinary skill in the art to which this invention pertains. Generally, the nomenclature used herein and the laboratory procedures of cell culture, molecular genetics, microbiology, organic chemistry, analytical chemistry and nucleic acid chemistry described below are those well-known and commonly employed in the art. Such techniques are well-known and described in numerous texts and reference works well known to those of skill in the art. Standard techniques, or modifications thereof, are used for chemical syntheses and chemical analyses. All patents, patent applications, articles and publications mentioned herein, both supra and infra, are hereby expressly incorporated herein by reference.

Although any suitable methods and materials similar or equivalent to those described herein find use in the practice of the present invention, some methods and materials are described herein. It is to be understood that this invention is not limited to the particular methodology, protocols, and reagents described, as these may vary, depending upon the context they are used by those of skill in the art. Accordingly, the terms defined immediately below are more fully described by reference to the invention as a whole.

It is to be understood that both the foregoing general description and the following detailed description are exemplary and explanatory only and are not restrictive of the present invention. The section headings used herein are for organizational purposes only and not to be construed as limiting the subject matter described. Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

### Abbreviations

The abbreviations used for the genetically encoded amino acids are conventional and are as follows:

| Amino Acid | Three-Letter Abbreviation | One-Letter Abbreviation |
|---|---|---|
| Alanine | Ala | A |
| Arginine | Arg | R |
| Asparagine | Asn | N |
| Aspartate | Asp | D |
| Cysteine | Cys | C |
| Glutamate | Glu | E |
| Glutamine | Gln | Q |
| Glycine | Gly | G |
| Histidine | HIS | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

When the three-letter abbreviations are used, unless specifically preceded by an “L” or a “D” or clear from the context in which the abbreviation is used, the amino acid may be in either the L- or D-configuration about α-carbon (C_{α}). For example, whereas “Ala” designates alanine without specifying the configuration about the α-carbon, “D-Ala” and “L-Ala” designate D-alanine and L-alanine, respectively. When the one-letter abbreviations are used, upper case letters designate amino acids in the L-configuration about the α-carbon and lower case letters designate amino acids in the D-configuration about the α-carbon. For example, “A” designates L-alanine and “a” designates D-alanine. When polypeptide sequences are presented as a string of one-letter or three-letter abbreviations (or mixtures thereof), the sequences are presented in the amino (N) to carboxy (C) direction in accordance with common convention.

The abbreviations used for the genetically encoding nucleosides are conventional and are as follows: adenosine (A); guanosine (G); cytidine (C); thymidine (T); and uridine (U). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When nucleic acid sequences are presented as a string of one-letter abbreviations, the sequences are presented in the 5′ to 3° direction in accordance with common convention, and the phosphates are not indicated.

### Definitions

In reference to the present invention, the technical and scientific terms used in the descriptions herein will have the meanings commonly understood by one of ordinary skill in the art, unless specifically defined otherwise. Accordingly, the following terms are intended to have the following meanings.

As used herein, the singular forms “a”, “an” and “the” include plural referents unless the context clearly indicates otherwise. Thus, for example, reference to “a polypeptide” includes more than one polypeptide.

Similarly, “comprise,” “comprises,” “comprising” “include,” “includes,” and “including” are interchangeable and not intended to be limiting. Thus, as used herein, the term “comprising” and its cognates are used in their inclusive sense (i.e., equivalent to the term “including” and its corresponding cognates).

It is to be further understood that where descriptions of various embodiments use the term “comprising,” those skilled in the art would understand that in some specific instances, an embodiment can be alternatively described using language “consisting essentially of” or “consisting of.”

The term “about” means an acceptable error for a particular value. In some instances “about” means within 0.05%, 0.5%, 1.0%, or 2.0%, of a given value range. In some instances, “about” means within 1, 2, 3, or 4 standard deviations of a given value.

“EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

“ATCC” refers to the American Type Culture Collection whose biorepository collection includes genes and strains.

“NCBI” refers to National Center for Biological Information and the sequence databases provided therein.

“Protein” “polypeptide,” and “peptide” are used interchangeably herein to denote a polymer of at least two amino acids covalently linked by an amide bond, regardless of length or post-translational modification (e.g., glycosylation or phosphorylation). Included within this definition are D- and L-amino acids, and mixtures of D- and L-amino acids, as well as polymers comprising D- and L-amino acids, and mixtures of D- and L-amino acids.

“Amino acids” are referred to herein by either their commonly known three-letter symbols or by the one-letter symbols recommended by IUPAC-IUB Biochemical Nomenclature Commission. Nucleotides, likewise, may be referred to by their commonly accepted single letter codes.

As used herein, “polynucleotide” and “nucleic acid’ refer to two or more nucleosides that are covalently linked together. The polynucleotide may be wholly comprised of ribonucleotides (i.e., RNA), wholly comprised of 2′ deoxyribonucleotides (i.e., DNA), or comprised of mixtures of ribo- and 2′ deoxyribonucleotides. While the nucleosides will typically be linked together via standard phosphodiester linkages, the polynucleotides may include one or more non-standard linkages. The polynucleotide may be single-stranded or double-stranded, or may include both single-stranded regions and double-stranded regions. Moreover, while a polynucleotide will typically be composed of the naturally occurring encoding nucleobases (i.e., adenine, guanine, uracil, thymine and cytosine), it may include one or more modified and/or synthetic nucleobases, such as, for example, inosine, xanthine, hypoxanthine, etc. In some embodiments, such modified or synthetic nucleobases are nucleobases encoding amino acid sequences

“Coding sequence” refers to that portion of a nucleic acid (e.g., a gene) that encodes an amino acid sequence of a protein.

As used herein, the terms “biocatalysis,” “biocatalytic,” “biotransformation,” and “biosynthesis” refer to the use of enzymes to perform chemical reactions on organic compounds.

“Glycosyltransferase” refers to a polypeptide having an enzymatic capability of transferring glycosyl residues from an activated sugar, for example a nucleotide diphosphate sugar or a phosphosugar, to monomeric and polymeric acceptor molecules.

“Phosphorylase” refers to a polypeptide having an enzymatic capability of cleaving glycosidic bonds using inorganic phosphate, releasing a phosphoglycoside and monomeric or polymeric product. In the reverse direction, a phosphorylase may act as a glycosyltransferase by transferring a glycosyl residue from a phosphoglycoside, for example glucose-1-phosphate, to monomeric and polymeric acceptor.

As used herein, “glycosylation” refers to the formation of a glycosidic linkage between a glycosyl residue and an acceptor molecule.

As used herein, “glucosylation” refers to the formation of a glycosidic linkage between a glucose residue and an acceptor molecule.

As used herein, “glycosyl” refers to an organic group that is a univalent free radical or substituent structure obtained by removing the hemiacetal hydroxyl group from the cyclic form of a monosaccharide, lower oligosaccharide or oligosaccharide derivative. Glycosyl groups react with inorganic acids (e.g., phosphoric acid) to form esters (e.g., glucose i-phosphate).

As used herein, “glycoside” refers to a molecule in which a carbohydrate (e.g., sugar) is bound to another functional group by a glycosidic bond. Glycosides can be hydrolyzed to produce a sugar and a non-sugar (i.e., aglycone) component.

As used herein, the term “steviol glycoside” refers to a glycoside of steviol, including but not limited to, naturally occurring steviol glycosides (e.g., stevioside, steviolmonoside, steviolbioside, rubusoside, dulcoside B, dulcoside A, rebaudioside B, rebaudioside G, rebaudioside C, rebaudioside F, rebaudioside A, rebaudioside I, rebaudioside E, rebaudioside H, rebaudioside L, rebaudioside K, rebaudioside J, rebaudioside M (also known as rebaudioside X), rebaudioside D, rebaudioside N, rebaudioside O), and synthetic steviol glycosides (e.g., enzymatically glucosylated steviol glycosides), and combinations thereof. The chemical structures of steviol and its glycosides are below (See, WO 2013/176738).

### Chemical Structures of Steviol and its Glycosides

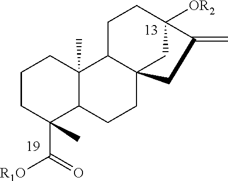

| | | |
|---|---|---|
| Steviol | H | H |
| Steviolmonoside | H | Glcβ1- |
| Steviol | Glcβ1- | H |
| monoglucosyl | | |
| ester | | |
| Rubusoside | Glcβ1- | Glcβ1- |
| Steviolbioside | H | Glcβ(1-2) Glcβ1- |
| Dulcoside A | G1cβ1- | Rhaα(1-2) Glcβ1- |
| Stevioside | G1cβ1- | Glcβ(1-2) Glcβ1- |
| Rebaudioside B | H | Glcβ(1-2)[Glcβ(1-3)] |
| | | Glcβ1- |
| Rebaudioside C | G1cβ1- | Rhaα(1-2)[Glcβ(1-3)] |
| | | Glcβ1- |
| Rebaudioside A | G1cβ1- | Glcβ(1-2)[Glcβ(1-3)] |
| | | Glcβ1- |
| Rebaudioside D | Glcβ(1-2) Glcβ1- | Glcβ(1-2)[Glcβ(1-3)] |
| | | Glcβ1- |
| Rebaudioside M | Glcβ(1-2)[Glcβ(1-3)] | Glcβ(1-2)[Glcβ(1-3)] |
| | Glcβ1- | Glcβ1- |

(Glc = glucose, Rha = rhamnose)

As used herein, “wild-type” and “naturally-occurring” refer to the form found in nature. For example a wild-type polypeptide or polynucleotide sequence is a sequence present in an organism that can be isolated from a source in nature and which has not been intentionally modified by human manipulation.

As used herein, “recombinant,” “engineered,” and “non-naturally occurring” when used with reference to a cell, nucleic acid, or polypeptide, refer to a material, or a material corresponding to the natural or native form of the material, that has been modified in a manner that would not otherwise exist in nature. In some embodiments, the cell, nucleic acid or polypeptide is identical a naturally occurring cell, nucleic acid or polypeptide, but is produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

The term “percent (%) sequence identity” is used herein to refer to comparisons among polynucleotides or polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted by any suitable method, including, but not limited to the local homology algorithm of Smith and Waterman (Smith and Waterman, Adv. Appl. Math., 2:482 [1981]), by the homology alignment algorithm of Needleman and Wunsch (Needleman and Wunsch, J. Mol. Biol., 48:443 [1970]), by the search for similarity method of Pearson and Lipman (Pearson and Lipman, Proc. Natl. Acad. Sci. USA 85:2444 [1988]), by computerized implementations of these algorithms (e.g., GAP, BESTFIT, FASTA, and TFASTA in the GCG Wisconsin Software Package), or by visual inspection, as known in the art. Examples of algorithms that are suitable for determining percent sequence identity and sequence similarity include, but are not limited to the BLAST and BLAST 2.0 algorithms, which are described by Altschul et al. (See Altschul et al., J. Mol. Biol., 215: 403-410 [1990]; and Altschul et al., Nucl. Acids Res., 3389-3402 [1977], respectively). Software for performing BLAST analyses is publicly available through the National Center for Biotechnology Informnnation website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (See, Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M:==5, N:=−4, and a comparison of both strands. For amino acid sequences, the BLASTP program uses as defaults a wordlength (W) of 3, an expectation (E) of 10, and the BLOSUM62 scoring matrix (See, Henikoff and Henikoff, Proc. Natl. Acad. Sci. USA 89:10915 [1989]). Exemplary determination of sequence alignment and % sequence identity can employ the BESTFIT or GAP programs in the GCG Wisconsin Software package (Accelrys, Madison Wis.), using default parameters provided.

As used herein, “reference sequence” refers to a defined sequence used as a basis for a sequence and/or activity comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, at least 100 residues in length or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptides are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence.

As used herein, “comparison window” refers to a conceptual segment of at least about 20 contiguous nucleotide positions or amino acid residues wherein a sequence may be compared to a reference sequence of at least 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. The comparison window can be longer than 20 contiguous residues, and includes, optionally 30, 40, 50, 100, or longer windows.

As used herein, “corresponding to,” “reference to,” and “relative to” when used in the context of the numbering of a given amino acid or polynucleotide sequence refer to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered glycosyltransferase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

As used herein, “substantial identity” refers to a polynucleotide or polypeptide sequence that has at least 80 percent sequence identity, at least 85 percent identity, at least between 89 to 95 percent sequence identity, or more usually, at least 99 percent sequence identity as compared to a reference sequence over a comparison window of at least 20 residue positions, frequently over a window of at least 30-50 residues, wherein the percentage of sequence identity is calculated by comparing the reference sequence to a sequence that includes deletions or additions which total 20 percent or less of the reference sequence over the window of comparison. In some specific embodiments applied to polypeptides, the term “substantial identity” means that two polypeptide sequences, when optimally aligned, such as by the programs GAP or BESTFIT using default gap weights, share at least 80 percent sequence identity, preferably at least 89 percent sequence identity, at least 95 percent sequence identity or more (e.g., 99 percent sequence identity). In some embodiments, residue positions that are not identical in sequences being compared differ by conservative amino acid substitutions.

As used herein, “amino acid difference” and “residue difference” refer to a difference in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based. For example, a “residue difference at position X93 as compared to SEQ ID NO:4” refers to a difference of the amino acid residue at the polypeptide position corresponding to position 93 of SEQ ID NO:4. Thus, if the reference polypeptide of SEQ ID NO:4 has a serine at position 93, then a “residue difference at position X93 as compared to SEQ ID NO:4” an amino acid substitution of any residue other than serine at the position of the polypeptide corresponding to position 93 of SEQ ID NO:4. In most instances herein, the specific amino acid residue difference at a position is indicated as “XnY” where “Xn” specified the corresponding position as described above, and “Y” is the single letter identifier of the amino acid found in the engineered polypeptide (i.e., the different residue than in the reference polypeptide). In some instances, the present invention also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. In some instances, a polypeptide of the present invention can include one or more amino acid residue differences relative to a reference sequence, which is indicated by a list of the specified positions where residue differences are present relative to the reference sequence. In some embodiments, where more than one amino acid can be used in a specific residue position of a polypeptide, the various amino acid residues that can be used are separated by a “/” (e.g., X307H/X307P or X307H/P). The slash may also be used to indicate multiple substitutions within a given variant (i.e., there is more than one substitution present in a given sequence. In some embodiments, the present invention includes engineered polypeptide sequences comprising one or more amino acid differences comprising conservative or non-conservative amino acid substitutions. In some additional embodiments, the present invention provides engineered polypeptide sequences comprising both conservative and non-conservative amino acid substitutions,

As used herein, “conservative amino acid substitution” refers to a substitution of a residue with a different residue having a similar side chain, and thus typically involves substitution of the amino acid in the polypeptide with amino acids within the same or similar defined class of amino acids. By way of example and not limitation, an amino acid with an aliphatic side chain is substituted with another aliphatic amino acid (e.g., alanine, valine, leucine, and isoleucine); an amino acid with an hydroxyl side chain is substituted with another amino acid with an hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basis side chain (e.g., lysine and arginine); an amino acid with an acidic side chain is substituted with another amino acid with an acidic side chain (e.g., aspartic acid or glutamic acid); and/or a hydrophobic or hydrophilic amino acid is replaced with another hydrophobic or hydrophilic amino acid, respectively.

As used herein, “non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

As used herein, “deletion” refers to modification to the polypeptide by removal of one or more amino acids from the reference polypeptide. Deletions can comprise removal of I or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an engineered glycosyltransferase enzyme. Deletions can be directed to the internal portions and/or terminal portions of the polypeptide. In various embodiments, the deletion can comprise a continuous segment or can be discontinuous.

As used herein, “insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

A “functional fragment” and “biologically active fragment” are used interchangeably herein to refer to a polypeptide that has an amino-terminal and/or carboxy-terminal deletion(s) and/or internal deletions, but where the remaining amino acid sequence is identical to the corresponding positions in the sequence to which it is being compared (e.g., a full-length engineered glycosyltransferase of the present invention) and that retains substantially all of the activity of the full-length polypeptide.

As used herein, “isolated polypeptide” refers to a polypeptide which is substantially separated from other contaminants that naturally accompany it (e.g., protein, lipids, and polynucleotides). The term embraces polypeptides which have been removed or purified from their naturally-occurring environment or expression system (e.g., within a host cell or via in vitro synthesis). The recombinant glycosyltransferase polypeptides may be present within a cell, present in the cellular medium, or prepared in various forms, such as lysates or isolated preparations. As such, in some embodiments, the recombinant glycosyltransferase polypeptides can be an isolated polypeptide.

As used herein, “substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. However, in some embodiments, the composition comprising glycosyltransferase comprises glycosyltransferase that is less than 50% pure (e.g., about 10%, about 20%, about 30%, about 40%, or about 50%) Generally, a substantially pure glycosyltransferase composition comprises about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated recombinant glycosyltransferase polypeptides are substantially pure polypeptide compositions.

As used herein, “improved enzyme property” refers to at least one improved property of an enzyme. In some embodiments, the present invention provides engineered glycosyltransferase polypeptides that exhibit an improvement in any enzyme property as compared to a reference glycosyltransferase polypeptide and/or a wild-type glycosyltransferase polypeptide, and/or another engineered glycosyltransferase polypeptide. Thus, the level of “improvement” can be determined and compared between various glycosyltransferase polypeptides, including wild-type, as well as engineered glycosyltransferases. Improved properties include, but are not limited, to such properties as increased protein expression, increased thermoactivity, increased thermostability, increased pH activity, increased stability, increased enzymatic activity, increased substrate specificity or affinity, increased specific activity, increased resistance to substrate or end-product inhibition, increased chemical stability, improved chemoselectivity, improved solvent stability, increased tolerance to acidic pH, increased tolerance to proteolytic activity (i.e., reduced sensitivity to proteolysis), reduced aggregation, increased solubility, and altered temperature profile.

As used herein, “increased enzymatic activity” and “enhanced catalytic activity” refer to an improved property of the engineered glycosyltransferase polypeptides, which can be represented by an increase in specific activity (e.g., product produced/time/weight protein) or an increase in percent conversion of the substrate to the product (e.g., percent conversion of starting amount of substrate to product in a specified time period using a specified amount of glycosyltransferase) as compared to the reference glycosyltransferase enzyme. Exemplary methods to determine enzyme activity are provided in the Examples. Any property relating to enzyme activity may be affected, including the classical enzyme properties of Kₘ, Vₘₐₓ or k_{cat}, changes of which can lead to increased enzymatic activity. Improvements in enzyme activity can be from about 1.1 fold the enzymatic activity of the corresponding wild-type enzyme, to as much as 2-fold, 5-fold, 10-fold, 20-fold, 25-fold, 50-fold, 75-fold, 100-fold, 150-fold, 200-fold or more enzymatic activity than the naturally occurring glycosyltransferase or another engineered glycosyltransferase from which the glycosyltransferase polypeptides were derived.

As used herein, “conversion” refers to the enzymatic conversion (or biotransfonnation) of a substrate(s) to the corresponding product(s). “Percent conversion” refers to the percent of the substrate that is converted to the product within a period of time under specified conditions. Thus, the “enzymatic activity” or “activity” of a glycosyltransferase polypeptide can be expressed as “percent conversion” of the substrate to the product in a specific period of time.

Enzymes with “generalist properties” (or “generalist enzymes”) refer to enzymes that exhibit improved activity for a wide range of substrates, as compared to a parental sequence. Generalist enzymes do not necessarily demonstrate improved activity for every possible substrate. In some embodiments, the present invention provides glycosyltransferase variants witth generalist properties, in that they demonstrate similar or improved activity relative to the parental gene for a wide range of sterically and electronically diverse substrates. In addition, the generalist enzymes provided herein were engineered to be improved across a wide range of diverse molecules to increase the production of metabolites/products.

The term “stringent hybridization conditions” is used herein to refer to conditions under which nucleic acid hybrids are stable. As known to those of skill in the art, the stability of hybrids is reflected in the melting temperature (Tₘ) of the hybrids. In general, the stability of a hybrid is a function of ion strength, temperature, G/C content, and the presence of chaotropic agents. The 7, values for polynucleotides can be calculated using known methods for predicting melting temperatures (See e.g.. Baldino et al., Meth. Enzymol., 168:761-777 [1989]; Bolton et al., Proc. Natl. Acad. Sci. USA 48:1390 [1962]; Bresslauer et al., Proc. Natl. Acad. Sci. USA 83:8893-8897 [1986]; Freier et al., Proc. Natl. Acad. Sci. USA 83:9373-9377 [1986]; Kierzek et al., Biochem., 25:7840-7846 [1986]; Rychlik et al., 1990, Nucl. Acids Res., 18:6409-6412 [1990] (erratum, Nucl. Acids Res., 19:698 [1991]); Sambrook et al., supra); Suggs et al., 1981, in Developmental Biology Using Purified Genes, Brown et al. [eds.], pp. 683-693, Academic Press, Cambridge, Mass. [1981]; and Wetmur, Crit. Rev. Biochem. Mol. Biol., 26:227-259 [1991]). In some embodiments, the polynucleotide encodes the polypeptide disclosed herein and hybridizes under defined conditions, such as moderately stringent or highly stringent conditions, to the complement of a sequence encoding an engineered glycosyltransferase enzyme of the present invention.

As used herein, “hybridization stringency” relates to hybridization conditions, such as washing conditions, in the hybridization of nucleic acids. Generally, hybridization reactions are performed under conditions of lower stringency, followed by washes of varying but higher stringency. The term “moderately stringent hybridization” refers to conditions that permit target-DNA to bind a complementary nucleic acid that has about 60% identity, preferably about 75% identity, about 85% identity to the target DNA, with greater than about 90% identity to target-polynucleotide. Exemplary moderately stringent conditions are conditions equivalent to hybridization in 50% formamide, 5×Denhart's solution, 5×SSPE, 0.2% SDS at 42° C., followed by washing in 0.2×SSPE, 0.2% SDS, at 42° C. “High stringency hybridization” refers generally to conditions that are about 10° C. or less from the thermal melting temperature Tₘ as determined under the solution condition for a defined polynucleotide sequence. In some embodiments, a high stringency condition refers to conditions that permit hybridization of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridization in conditions equivalent to 50% formamide, 5×Denhart's solution, 5×SSPE, 0.2% SDS at 42° C., followed by washing in 0.1×SSPE, and 0.1% SDS at 65° C. Another high stringency condition is hybridizing in conditions equivalent to hybridizing in 5×SSC containing 0.1% (w:v) SDS at 65° C. and washing in 0.1×SSC containing 0.1% SDS at 65° C. Other high stringency hybridization conditions, as well as moderately stringent conditions, are described in the references cited above.

As used herein, “codon optimized” refers to changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the glycosyltransferase enzymes may be codon optimized for optimal production in the host organism selected for expression.

As used herein, “preferred,” “optimal,” and “high codon usage bias” codons when used alone or in combination refer interchangeably to codons that are used at higher frequency in the protein coding regions than other codons that code for the same amino acid. The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression. A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (See e.g., GCG CodonPreference, Genetics Computer Group Wisconsin Package; CodonW, Peden, University of Nottingham; McInerney, Bioinform., 14:372-73 [1998]; Stenico et al., Nucl. Acids Res., 222437-46 [1994]; Wright, Gene 87:23-29 [1990]). Codon usage tables are available for many different organisms (See e.g., Wada et al., Nucl. Acids Res., 20:2111-2118 [1992]; Nakamura et al., Nucl. Acids Res., 28:292 [2000]; Duret, et al., supra; Henaut and Danchin, in Escherichia coli and Salmonella, Neidhardt, et al. (eds.), ASM Press, Washington D.C., p. 2047-2066 [1996]). The data source for obtaining codon usage may rely on any available nucleotide sequence capable of coding for a protein. These data sets include nucleic acid sequences actually known to encode expressed proteins (e.g., complete protein coding sequences-CDS), expressed sequence tags (ESTS), or predicted coding regions of genomic sequences (See e.g., Mount, Bioinformatics: Sequence and Genome Analysis, Chapter 8, Cold Spring Harbor Laboratory Press, Cold Spring Harbor, N.Y. [2001]; Uberbacher, Meth. Enzymol., 266:259-281 [1996]; and Tiwari et al., Comput. Appl. Biosci., 13:263-270 [1997]).

As used herein, “control sequence” includes all components, which are necessary or advantageous for the expression of a polynucleotide and/or polypeptide of the present invention. Each control sequence may be native or foreign to the nucleic acid sequence encoding the polypeptide. Such control sequences include, but are not limited to, a leader, polyadenylation sequence, propeptide sequence, promoter sequence, signal peptide sequence, initiation sequence and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide.

“Operably linked” is defined herein as a configuration in which a control sequence is appropriately placed (i.e., in a functional relationship) at a position relative to a polynucleotide of interest such that the control sequence directs or regulates the expression of the polynucleotide and/or polypeptide of interest.

“Promoter sequence” refers to a nucleic acid sequence that is recognized by a host cell for expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

The phrase “suitable reaction conditions” refers to those conditions in the enzymatic conversion reaction solution (e.g., ranges of enzyme loading, substrate loading, temperature, pH, buffers, co-solvents, etc.) under which a glycosyltransferase polypeptide of the present invention is capable of converting a substrate to the desired product compound. Some exemplary “suitable reaction conditions” are provided herein.

As used herein, “loading,” such as in “compound loading” or “enzyme loading” refers to the concentration or amount of a component in a reaction mixture at the start of the reaction.

As used herein, “substrate” in the context of an enzymatic conversion reaction process refers to the compound or molecule acted on by the glycosyitransferase polypeptide.

As used herein, “product” in the context of an enzymatic conversion process refers to the compound or molecule resulting from the action of the glycosyltransferase polypeptide on a substrate.

As used herein the terra “culturing” refers to the growing of a population of microbial cells under any suitable conditions (e.g., using a liquid, gel or solid medium).

Recombinant polypeptides can be produced using any suitable methods known in the art. Genes encoding the wild-type polypeptide of interest can be cloned in vectors, such as plasmids, and expressed in desired hosts, such as E. coli, etc. Variants of recombinant polypeptides can be generated by various methods known in the art. Indeed, there is a wide variety of different mutagenesis techniques well known to those skilled in the art. In addition, mutagenesis kits are also available from many commercial molecular biology suppliers. Methods are available to make specific substitutions at defined amino acids (site-directed), specific or random mutations in a localized region of the gene (regio-specific), or random mutagenesis over the entire gene (e.g., saturation mutagenesis). Numerous suitable methods are known to those in the art to generate enzyme variants, including but not limited to site-directed mutagenesis of single-stranded DNA or double-stranded DNA using PCR, cassette mutagenesis, gene synthesis, error-prone PCR, shuffling, and chemical saturation mutagenesis, or any other suitable method known in the art. Non-limiting examples of methods used for DNA and protein engineering are provided in the following patents: U.S. Pat. Nos. 6,117,679; 6,420,175; 6,376,246; 6,586,182; 7,747,391; 7,747,393; 7,783,428; and 8,383,346. After the variants are produced, they can be screened for any desired property (e.g., high or increased activity, or low or reduced activity, increased thermal activity, increased thermal stability, and/or acidic pH stability, etc.). In some embodiments, “recombinant glycosyltransferase polypeptides” (also referred to herein as “engineered glycosyltransferase polypeptides,” “variant glycosyltransferase enzymes,” and “glycosyltransferase variants”) find use.

As used herein, a “vector” is a DNA construct for introducing a DNA sequence into a cell. In some embodiments, the vector is an expression vector that is operably linked to a suitable control sequence capable of effecting the expression in a suitable host of the polypeptide encoded in the DNA sequence. In some embodiments, an “expression vector” has a promoter sequence operably linked to the DNA sequence (e.g., transgene) to drive expression in a host cell, and in some embodiments, also comprises a transcription terminator sequence.

As used herein, the term “expression” includes any step involved in the production of the polypeptide including, but not limited to, transcription, post-transcriptional modification, translation, and post-translational modification. In some embodiments, the term also encompasses secretion of the polypeptide from a cell.

As used herein, the term “produces” refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

As used herein, an amino acid or nucleotide sequence (e.g., a promoter sequence, signal peptide, terminator sequence, etc.) is “heterologous” to another sequence with which it is operably linked if the two sequences are not associated in nature. For example a “heterologous polynucleotide” is any polynucleotide that is introduced into a host cell by laboratory techniques, and includes polynucleotides that are removed from a host cell, subjected to laboratory manipulation, and then reintroduced into a host cell.

As used herein, the terms “host cell” and “host strain” refer to suitable hosts for expression vectors comprising DNA provided herein (e.g., the polynucleotides encoding the glycosyltransferase variants). In some embodiments, the host cells are prokaryotic or eukaryotic cells that have been transformed or transfected with vectors constructed using recombinant DNA techniques as known in the art.

The term “analogue” means a polypeptide having more than 70% sequence identity but less than 100% sequence identity (e.g., more than 75%, 78%, 80%, 83%, 85%, 88%, 90%, 91%, 92%, 93%, 94%, 95%, 96%, 97%, 98%, 99% sequence identity) with a reference polypeptide. In some embodiments, analogues means polypeptides that contain one or more non-naturally occurring amino acid residues including, but not limited, to homoarginine, omrithine and norvaline, as well as naturally occurring amino acids. In some embodiments, analogues also include one or more D-amino acid residues and non-peptide linkages between two or more amino acid residues.

The term “effective amount” means an amount sufficient to produce the desired result. One of general skill in the art may determine what the effective amount by using routine experimentation.

The terms “isolated” and “purified” are used to refer to a molecule (e.g., an isolated nucleic acid, polypeptide, etc.) or other component that is removed from at least one other component with which it is naturally associated. The term “purified” does not require absolute purity, rather it is intended as a relative definition.

“Stereoselectivity” refers to the preferential formation in a chemical or enzymatic reaction of one stereoisomer over another. Stereoselectivity can be partial, where the formation of one stereoisomer is favored over the other, or it may be complete where only one stereoisomer is formed. When the stereoisomers are enantiomers, the stereoselectivity is referred to as enantioselectivity, the fraction (typically reported as a percentage) of one enantiomer in the sum of both. It is commonly alternatively reported in the art (typically as a percentage) as the enantiomeric excess (e.e.) calculated therefrom according to the formula [major enantiomer−minor enantiomer]/[major enantiomer+minor enantiomer]. Where the stereoisomers are diastereoisomers, the stereoselectivity is referred to as diastereoselectivity, the fraction (typically reported as a percentage) of one diastereomer in a mixture of two diastereomers, commonly alternatively reported as the diastereomeric excess (d.e.). Enantiomeric excess and diastereomeric excess are types of stereomeric excess.

As used herein, the terms “regioselectivity” and “regioselective reaction” refer to a reaction in which one direction of bond making or breaking occurs preferentially over all other possible directions. Reactions can completely (100%) regioselective if the discrimination is complete, substantially regioselective (at least 75%), or partially regioselective (x %, wherein the percentage is set dependent upon the reaction of interest), if the product of reaction at one site predominates over the product of reaction at other sites, for example, preferential formation of the product compound (2) (i.e., 2S,3S0-hydroxypipecolic acid over the undesired product (2S,5S)-hydroxypipecolic acid.

As used herein, “thermostable” refers to a glycosyltransferase polypeptide that maintains similar activity (more than 60% to 80% for example) after exposure to elevated temperatures (e.g., 40-80° C.) for a period of time (e.g., 0.5-24 h) compared to the wild-type enzyme exposed to the same elevated temperature.

As used herein, “solvent stable” refers to a glycosyltransferase polypeptide that maintains similar activity (more than e.g., 60% to 80%) after exposure to varying concentrations (e.g., 5-99%) of solvent (ethanol, isopropyl alcohol, dimethylsulfoxide (DMSO), tetrahydrofuran, 2-methyltetrahydrofuran, acetone, toluene, butyl acetate, methyl tert-butyl ether, etc.) for a period of time (e.g., 0.5-24 h) compared to the wild-type enzyme exposed to the same concentration of the same solvent.

As used herein, “thermo- and solvent stable” refers to a glycosyltransferase polypeptide that is both thermostable and solvent stable.

As used herein, “reductant” refers to a compound or agent capable of converting Fe⁺³ to Fe⁺². An exemplary reductant is ascorbic acid, which is generally in the form of L-ascorbic acid.

“Alkyl” refers to saturated hydrocarbon groups of from 1 to 18 carbon atoms inclusively, either straight chained or branched, more preferably from 1 to 8 carbon atoms inclusively, and most preferably 1 to 6 carbon atoms inclusively. An alkyl with a specified number of carbon atoms is denoted in parenthesis (e.g., (C₁—C)alkyl refers to an alkyl of I to 6 carbon atoms).

“Alkenyl” refers to hydrocarbon groups of from 2 to 12 carbon atoms inclusively, either straight or branched containing at least one double bond but optionally containing more than one double bond.

“Alkynyl” refers to hydrocarbon groups of from 2 to 12 carbon atoms inclusively, either straight or branched containing at least one triple bond but optionally containing more than one triple bond, and additionally optionally containing one or more double bonded moieties.

“Alkylene” refers to a straight or branched chain divalent hydrocarbon radical having from 1 to 18 carbon atoms inclusively, more preferably from 1 to 8 carbon atoms inclusively, and most preferably 1 to 6 carbon atoms inclusively, optionally substituted with one or more suitable substituents. Exemplary “alkylenes” include, but are not limited to, methylene, ethylene, propylene, butylene, and the like.

“Alkenylene” refers to a straight or branched chain divalent hydrocarbon radical having 2 to 12 carbon atoms inclusively and one or more carbon-carbon double bonds, more preferably from 2 to 8 carbon atoms inclusively, and most preferably 2 to 6 carbon atoms inclusively, optionally substituted with one or more suitable substituents.

“Heteroalkyl, “heteroalkenyl,” and heteroalkynyl,” refer respectively, to alkyl, alkenyl and alkynyl as defined herein in which one or more of the carbon atoms are each independently replaced with the same or different heteroatoms or heteroatomic groups. Heteroatoms and/or heteroatomic groups which can replace the carbon atoms include, but are not limited to, —O—, —S—, —S—O—, —NR^{γ}—, —PH—, —S(O)—, —S(O)2-, —S(O)NR^{γ}—, —S(O)2NR^{γ}—, and the like, including combinations thereof, where each W is independently selected from hydrogen, alkyl, cycloalkyl, heterocycloalkyl, aryl, and heteroaryl.

“Aryl” refers to an unsaturated aromatic carbocyclic group of from 6 to 12 carbon atoms inclusively having a single ring (e.g., phenyl) or multiple condensed rings (e.g., naphthyl or anthryl). Exemplary aryls include phenyl, pyridyl, naphthyl and the like.

“Arylalkyl” refers to an alkyl substituted with an aryl (i.e., aryl-alkyl-groups), preferably having from 1 to 6 carbon atoms inclusively in the alkyl moiety and from 6 to 12 carbon atoms inclusively in the aryl moiety. Such arylalkyl groups are exemplified by benzyl, phenethyl and the like.

“Aryloxy” refers to —OR groups, where R is an aryl group, which can be optionally substituted.

“Cycloalkyl” refers to cyclic alkyl groups of from 3 to 12 carbon atoms inclusively having a single cyclic ring or multiple condensed rings which can be optionally substituted with from 1 to 3 alkyl groups. Exemplary cycloalkyl groups include, but are not limited to, single ring structures such as cyclopropyl, cyclobutyl, cyclopentyl, cyclooctyl, I-methylcyclopropyl, 2-methylcyclopentyl, 2-methylcyclooctyl, and the like, or multiple ring structures, including bridged ring systems, such as adamantyl, and the like.

“Cycloalkylalkyl” refers to an alkyl substituted with a cycloalkyl (i.e., cycloalkyl-alkyl-groups), preferably having from 1 to 6 carbon atoms inclusively in the alkyl moiety and from 3 to 12 carbon atoms inclusively in the cycloalkyl moiety. Such cycloalkylalkyl groups are exemplified by cyclopropylmethyl, cyclohexylethyl and the like.

“Amino” refers to the group —NH₂. Substituted amino refers to the group —NHR^{η}, NR^{η}R^{η}, and NR^{η}R^{η}R^{η}, where each R^{η} is independently selected from substituted or unsubstituted alkyl, cycloalkyl, cycloheteroalkyl, alkoxy, aryl, heteroaryl, heteroarylalkyl, acyl, alkoxycarbonyl, sulfanyl, sulfinyl, sulfonyl, and the like. Typical amino groups include, but are limited to, dimethylamino, diethylamino, trimethylammonium, triethylammonium, methylysulfonylamino, furanyl-oxy-sulfamino, and the like.

“Aminoalkyl” refers to an alkyl group in which one or more of the hydrogen atoms are replaced with one or more amino groups, including substituted amino groups.

“Aminocarbonyl” refers to —C(O)NH₂. Substituted aminocarbonyl refers to —C(O)NR^{η}R^{η}, where the amino group NR^{η}R^{η} is as defined herein.

“Oxy” refers to a divalent group —O—, which may have various substituents to form different oxy groups, including ethers and esters.

“Alkoxy” or “alkyloxy” are used interchangeably herein to refer to the group —OR, wherein RA is an alkyl group, including optionally substituted alkyl groups.

“Carboxy” refers to —COOH.

“Carbonyl” refers to —C(O)—, which may have a variety of substituents to form different carbonyl groups including acids, acid halides, aldehydes, amides, esters, and ketones.

“Carboxyalkyl” refers to an alkyl in which one or more of the hydrogen atoms are replaced with one or more carboxy groups.

“Aminocarbonylalkyl” refers to an alkyl substituted with an aminocarbonyl group, as defined herein.

“Halogen” or “halo” refers to fluoro, chloro, bromo and iodo.

“Haloalkyl” refers to an alkyl group in which one or more of the hydrogen atoms are replaced with a halogen. Thus, the term “haloalkyl” is meant to include monohaloalkyls, dihaloalkyls, trihaloalkyls, etc. up to perhaloalkyls. For example, the expression “(C₁-C₂) haloalkyl” includes 1-fluoromethyl, difluoromethyl, trifluoromethyl, 1-fluoroethyl, 1,1-difluoroethyl, 1,2-difluoroethyl, 1,1,1 trifluoroethyl, perfluoroethyl, etc.

“Hydroxy” refers to —OH.

“Hydroxyalkyl” refers to an alkyl group in which in which one or more of the hydrogen atoms are replaced with one or more hydroxy groups.

“Thiol” or “sulfanyl” refers to —SH. Substituted thiol or sulfanyl refers to —S—R^{η}, where R^{η} is an alkyl, aryl or other suitable substituent.

“Alkylthio” refers to —SR^{ζ}, where R^{ζ} is an alkyl, which can be optionally substituted. Typical alkylthio group include, but are not limited to, methylthio, ethylthio, n-propylthio, and the like.

“Alkylthioalkyl” refers to an alkyl substituted with an alkylthio group, —SR^{ζ}, where R^{ζ} is an alkyl, which can be optionally substituted.

“Sulfonyl” refers to —SO₂—. Substituted sulfonyl refers to —SO₂—R^{η}, where R^{η} is an alkyl, aryl or other suitable substituent.

“Alkylsulfonyl” refers to —SO₂—R^{ζ}, where R^{ζ} is an alkyl, which can be optionally substituted. Typical alkylsulfonyl groups include, but are not limited to, methylsulfonyl, ethylsulfonyl, n-propylsulfonyl, and the like.

“Alkylsulfonylalkyl” refers to an alkyl substituted with an alkylsulfonyl group, —SO₂—R^{ζ}, where R^{ζ} is an alkyl, which can be optionally substituted.

“Heteroaryl” refers to an aromatic heterocyclic group of from 1 to 10 carbon atoms inclusively and 1 to 4 heteroatorms inclusively selected from oxygen, nitrogen and sulfur within the ring. Such heteroaryl groups can have a single ring (e.g., pyridyl or furyl) or multiple condensed rings (e.g., indolizinyl or benzothienyl).

“Heteroarylalkyl” refers to an alkyl substituted with a heteroaryl (i.e., heteroaryl-alkyl-groups), preferably having from 1 to 6 carbon atoms inclusively in the alkyl moiety and from 5 to 12 ring atoms inclusively in the heteroaryl moiety. Such heteroarylalkyl groups are exemplified by pyridylmethyl and the like.

“Heterocycle”, “heterocyclic” and interchangeably “heterocycloalkyl” refer to a saturated or unsaturated group having a single ring or multiple condensed rings, from 2 to 10 carbon ring atoms inclusively and from 1 to 4 hetero ring atoms inclusively selected from nitrogen, sulfur or oxygen within the ring. Such heterocyclic groups can have a single ring (e.g., piperidinyl or tetrahydrofuryl) or multiple condensed rings (e.g., indolinyl, dihydrobenzofuran or quinuclidinyl). Examples of heterocycles include, but are not limited to, furan, thiophene, thiazole, oxazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, pyrrolidine, indoline and the like.

“Heterocycloalkylalkyl” refers to an alkyl substituted with a heterocycloalkyl (i.e., heterocycloalkyl-alkyl-groups), preferably having from 1 to 6 carbon atoms inclusively in the alkyl moiety and from 3 to 12 ring atoms inclusively in the heterocycloalkyl moiety.

“Membered ring” is meant to embrace any cyclic structure. The number preceding the term “membered” denotes the number of skeletal atoms that constitute the ring. Thus, for example, cyclohexyl, pyridine, pyran and thiopyran are 6-membered rings and cyclopentyl, pyrrole, furan, and thiophene are 5-membered rings.

“Fused bicyclic ring” as used herein refers to both unsubstituted and substituted carbocyclic and/or heterocyclic ring moieties having 5 to 8 atoms in each ring, the rings having 2 common atoms.

Unless otherwise specified, positions occupied by hydrogen in the foregoing groups can be further substituted with substituents exemplified by, but not limited to, hydroxy, oxo, nitro, methoxy, ethoxy, alkoxy, substituted alkoxy, trifluoromethoxy, haloalkoxy, fluoro, chloro, bromo, iodo, halo, methyl, ethyl, propyl, butyl, alkyl, alkenyl, alkynyl, substituted alkyl, trifluorornethyl, haloalkyl, hydroxyalkyl, alkoxyalkyl, thio, alkylthio, acyl, carboxy, alkoxycarbonyl, carboxarnido, substituted carboxamido, alkylsulfonyl, alkylsulfinyl, alkylsulfonylamino, sulfonamido, substituted sulfonarmido, cyano, amino, substituted amino, alkylamino, dialkylamino, aminoalkyl, acylamino, amidino, amidoximo, hydroxamoyl, phenyl, aryl, substituted aryl, aryloxy, arylalkyl, arylalkenyl, arylalkynyl, pyridyl, imidazolyl, heteroaryl, substituted heteroaryl, heteroaryloxy, heteroarylalkyl, heteroarylalkenyl, heteroarylalkynyl, cyclopropyl cyclobutyl cyclopentyl, cyclohexyl, cycloalkyl, cycloalkenyl, cycloalkylalkyl, substituted cycloalkyl, cycloalkyloxy, pyrrolidinyl, piperidinyl, morpholino, heterocycle, (heterocycle)oxy, and (heterocycle)alkyl; and preferred heteroatorns are oxygen, nitrogen, and sulfur. It is understood that where open valences exist on these substituents they can be further substituted with alkyl, cycloalkyl aryl, heteroaryl, and/or heterocycle groups, that where these open valences exist on carbon they can be further substituted by halogen and by oxygen-, nitrogen-, or sulfur-bonded substituents, and where multiple such open valences exist, these groups can be joined to form a ring, either by direct formation of a bond or by formation of bonds to a new heteroatom, preferably oxygen, nitrogen, or sulfir. It is further understood that the above substitutions can be made provided that replacing the hydrogen with the substituent does not introduce unacceptable instability to the molecules of the present invention, and is otherwnise chemically reasonable.

As used herein, “optional” and “optionally” mean that the subsequently described event or circumstance may or may not occur, and that the description includes instances where the event or circumstance occurs and instances in which it does not. One of ordinary skill in the art would understand that with respect to any molecule described as containing one or more optional substituents, only sterically practical and/or synthetically feasible compounds are meant to be included. “Optionally substituted” refers to all subsequent modifiers in a term or series of chemical groups. For example, in the term “optionally substituted arylalkyl, the “alkyl” portion and the “aryl” portion of the molecule may or may not be substituted, and for the series “optionally substituted alkyl, cycloalkyl, aryl and heteroaryl,” the alkyl cycloalkyl, aryl, and heteroaryl groups, independently of the others, may or may not be substituted.

### Glycosylation

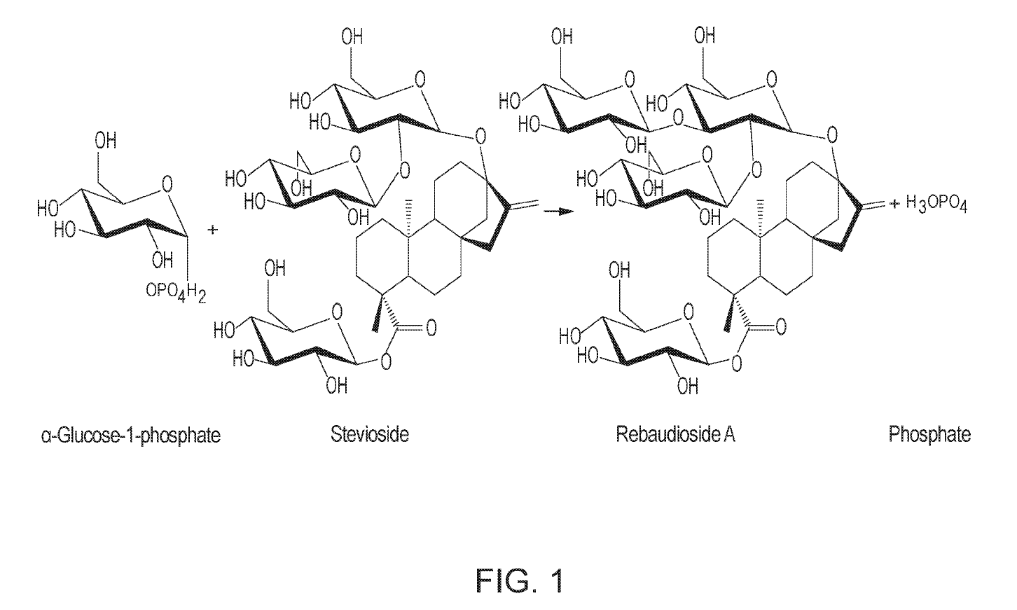

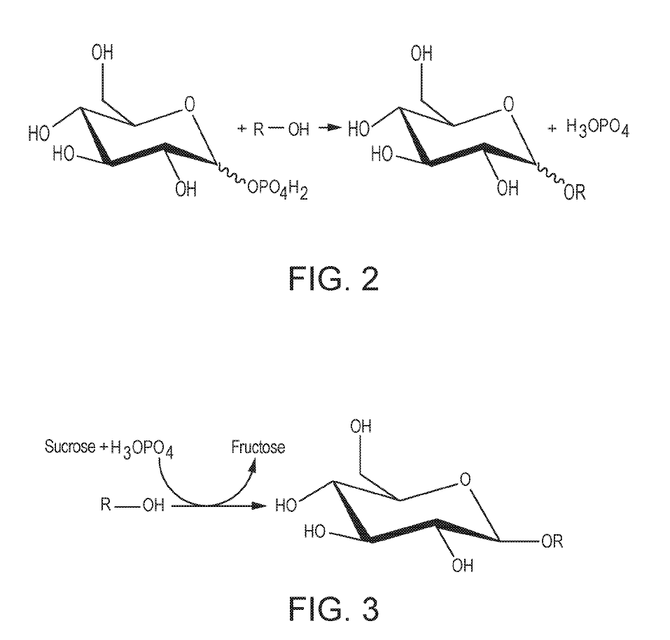

Glycosylation can alter many properties of natural and synthetic products including stability, pharnnacodynamics, solubility, and membrane transport. Many molecules, including many secondary metabolites with antimicrobial, antitumor, natural sweetness properties, etc., comprise non-ribosomal peptide, polyketide, or terpenoid backbones modified with β-glycosidic linkages. Many of the diterpene glycosides extracted from the plant, Stevia rebaudiana Bertoni, contain β-linked glucose molecules. Naturally, these molecules are glycosylated in vivo using UDP-glucose dependent glycosyl transferase enzymes. However, when used in vitro, the UDP-glucose can be prohibitively expensive and/or unavailable. In the present invention a new reaction scheme (See, FIG. 2) is provided, in which two different enzyme classes are used to transfer the glucose moiety from α-glucose-1-phosphate to a sample substrate (e.g., stevioside), to produce one or more β-glucose linked products. In some embodiments, the natural stevioside UDP-glucose dependent glycosyltransferase is shown to have activity, using α-glucose-1-phosphate to form rebaudioside A (See, FIG. 1). In the some additional embodiments, a laminaribiose phosphorylase acts in the reverse direction to form a β-glucose linked stevioside compound and inorganic phosphate from α-glucose-1-phosphate and stevioside.

Thus, glycosylation finds use in the production of natural sweeteners, such as those derived from the sweet herb, Stevia rebaudiana Bertoni. As indicated above, this plant produces a number of diterpene glycosides which feature high intensity sweetness and sensory properties superior to those of many other high potency sweeteners. The above-mentioned sweet glycosides, have a common aglycone (i.e., steviol), and differ by the number and type of carbohydrate residues at the C13 and C19 positions. Steviol glycosides differ from each other not only in their molecular structure, but also by their taste properties. Usually, stevioside is reported to be 110-270 times sweeter than sucrose, while rebaudioside A is reported to be between 150 and 320 times sweeter than sucrose, and rebaudioside C is reported to be between 40-60 times sweeter than sucrose. Of these common compounds, rebaudioside A has the least astringent, the least bitter, and the least persistent aftertaste. Thus, it has the most favorable sensory attributes of the major steviol glycosides. However, rebaudioside A only constitutes a minor fraction (2-10%) of total glycosides isolated from Stevia rebaudiana Bertoni, with other compounds including stevioside (2-10%) and Rebaudioside C (1-2%) making up the rest.

Changing the glycosylation pattern of some substrates finds use in either simplifying purification and/or to convert less desirable molecules (e.g., stevioside) to more desirable compounds (e.g., rebaudioside A). In some cases, glycosylation is achieved through chemical synthesis methods. However, these methods typically require multiple synthetic steps with undesirable chemicals and processes and can result in mixed products (e.g., with linkages in incorrect positions and/or with undesired anomeric configurations).

In contrast, glycosylating enzymes can be active under mild conditions and can confer high positional selectivity and stereospecificity in a single step. Many naturally derived glycosylated metabolites are generated in vivo using glycosyltransferase (GT) enzymes which transfer sugar moieties from various sugar nucleotides. When used in vitro processes however, the sugar nucleotide donors that these enzymes require can be prohibitively expensive and may not be available at scale. Therefore, there is a need for an enzyme capable of producing glycosvlated molecules using less expensive and/or more convenient sugar donors.

### Engineered Glycosyltransferase Polypeptides

The present invention provides polypeptides having glycosyltransferase activity, polynucleotides encoding the polypeptides, methods of preparing the polypeptides, and methods for using the polypeptides. Where the description relates to polypeptides, it is to be understood that it also describes the polynucleotides encoding the polypeptides.

The suitable reaction conditions under which the above-described improved properties of the engineered polypeptides carry out the transferase reaction can be determined with respect to concentrations or amounts of polypeptide, substrate, co-substrate, transition metal cofactor, reductant, buffer, co-solvent, pH, conditions including temperature and reaction time, and/or conditions with the polypeptide immobilized on a solid support, as further described below and in the Examples.

In some embodiments, exemplary engineered polypeptides having glycosyltransferase activity with improved properties, particularly in the conversion of steviol glycosides to further glycosylated steviol glycosides, comprise an amino acid sequence that has one or more residue differences as compared to SEQ ID NO: 2, 4, 6, and/or 8.

Residue differences at these other residue positions can provide for additional variations in the amino acid sequence without adversely affecting the ability of the polypeptide to carry out the transferase reaction. In some embodiments, the sequence further comprises 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, or 1-50 residue differences at other amino acid residue positions as compared to the SEQ ID NO: 2, 4, 6, and/or 8. In some embodiments, the number of amino acid residue differences as compared to the reference sequence can be 1, 2, 3, 4, 5, 6, 7, 8, 9, 10, 11, 12, 13, 14, 15, 16, 17, 18, 19, 20, 21, 22, 23, 24, 25, 30, 30, 35, 40, 45 or 50 residue positions. In some embodiments, the number of amino acid residue differences as compared to the reference sequence can be 1, 2, 3, 4, 5, 6, 7, 8, 9, 10, 11, 12, 13, 14, 15, 16, 18, 20, 21, 22, 23, 24, or 25 residue positions. The residue difference at these other positions can be conservative changes or non-conservative changes. In some embodiments, the residue differences can comprise conservative substitutions and non-conservative substitutions as compared to the naturally occurring glycosyltransferase polypeptide of SEQ ID NO: 2, 4, 6, and/or 8.

In some embodiments, the present invention also provides engineered polypeptides that comprise a fragment of any of the engineered glycosyltransferase polypeptides described herein that retains the functional activity and/or improved property of that engineered glycosyltransferase. Accordingly, in some embodiments, the present invention provides a polypeptide fragment capable of the transferase reaction under suitable reaction conditions, wherein the fragment comprises at least about 80%, 90%, 95%, 96%, 97%, 98%, or 99% of a fuill-length amino acid sequence of a glycosyltransferase polypeptide of the present invention, such as the naturally occurring glycosyltransferase polypeptide of 2, 4, 6, and/or 8.

In some embodiments, the engineered glycosyltransferase polypeptide can have an amino acid sequence comprising a deletion of any one of the glycosyltransferase polypeptides described herein, such as the naturally occurring glycosyltransferase polypeptide of SEQ ID NO: 2, 4, 6, and/or 8.

Thus, for each and every embodiment of the engineered glycosyltransferase polypeptides of the invention, the amino acid sequence can comprise deletions of one or more amino acids, 2 or more amino acids, 3 or more amino acids, 4 or more amino acids, 5 or more amino acids, 6 or more amino acids, 8 or more amino acids, 10 or more amino acids, 15 or more amino acids, or 20 or more amino acids, up to 10% of the total number of amino acids, up to 10% of the total number of amino acids, up to 20% of the total number of amino acids, or up to 30% of the total number of amino acids of the glycosyltransferase polypeptides, where the associated functional activity and/or improved properties of the engineered glycosyltransferase described herein are maintained. In some embodiments, the deletions can comprise 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residues. In some embodiments, the number of deletions can be 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residues. In some embodiments, the deletions can comprise deletions of 1, 2, 3, 4, 5, 6, 7, 8, 9, 10, 11, 12, 13, 14, 15, 16, 18, 20, 21, 22, 23, 24, or 25 amino acid residues.

In some embodiments, the engineered glycosyltransferase polypeptide herein can have an amino acid sequence comprising an insertion as compared to any one of the glycosyltransferase polypeptides described herein, such as the naturally occurring glycosyltransferase polypeptide of SEQ ID NO: 2, 4, 6, and/or 8. Thus, some embodiments of the glycosyltransferase polypeptides of the present invention, the insertions can comprise one or more amino acids, 2 or more amino acids, 3 or more amino acids, 4 or more amino acids, 5 or more amino acids, 6 or more amino acids, 8 or more amino acids, 10 or more amino acids, 15 or more amino acids, 20 or more amino acids, 30 or more amino acids, 40 or more amino acids, or 50 or more amino acids, where the associated functional activity and/or improved properties of the engineered glycosyltransferase described herein is maintained. The insertions can be to amino or carboxy terminus, or internal portions of the glycosyltransferase polypeptide.

In some embodiments, the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residue deletions, insertions and/or substitutions. In some embodiments, the number of amino acid sequence has optionally 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 18, 20, 21, 22, 23, 24, or 25 amino acid residue deletions, insertions and/or substitutions. In some embodiments, the substitutions can be conservative or non-conservative substitutions.

In the above embodiments, the suitable reaction conditions for the engineered polypeptides are provided in the Examples.

In some embodiments, the polypeptides of the present invention are fusion polypeptides in which the engineered polypeptides are fused to other polypeptides, such as, by way of example and not limitation, antibody tags (e.g., myc epitope), purification sequences (e.g., His tags for binding to metals), and cell localization signals (e.g., secretion signals). Thus, the engineered polypeptides described herein can be used with or without fusions to other polypeptides.

It is to be understood that the polypeptides described herein are not restricted to the genetically encoded amino acids. In addition to the genetically encoded amino acids, the polypeptides described herein may be comprised, either in whole or in part, of naturally occurring and/or synthetic non-encoded amino acids. Certain commonly encountered non-encoded amino acids of which the polypeptides described herein may be comprised include, but are not limited to: the D-stereomers of the genetically-encoded amino acids; 2,3-diaminopropionic acid (Dpr); α-aminoisobutyric acid (Aib); ε-aminohexanoic acid (Aha); δ-aminovaleric acid (Ava); N-methylglycine or sarcosine (MeGly or Sar); ornithine (Orn); citrulline (Cit); t-butylalanine (Bua); t-butylglycine (Bug); N-methylisoleucine (Melle); phenylglycine (Phg); cyclohexylalanine (Cha); norleucine (Nle); naphthylalanine (Nal); 2-chlorophenylalanine (Ocf); 3-chlorophenylalanine (Mcf); 4-chlorophenylalanine (Pcf); 2-fluorophenylalanine (Off); 3-fluorophenylalanine (Mft); 4-fluorophenylalanine (Pff); 2-bromophenylalanine (Obf); 3-bromophenylalanine (Mbf); 4-bromophenylalanine (Pbf); 2-methylphenylalanine (Omf); 3-methylphenylalanine (Mmf); 4-methylphenylalanine (Pmf); 2-nitrophenylalanine (Onf); 3-nitrophenylalanine (Mnf); 4-nitrophenylalanine (Pnf); 2-cyanophenylalanine (Ocf); 3-cyanophenylalanine (Mct); 4-cyanophenylalanine (Pcf); 2-trifluoromethylphenylalanine (Otf); 3-trifluoromethylphenylalanine (Mtf); 4-trifluoromethylphenylaanine (Ptft); 4-aminophenylalanine (Paf); 4-iodophenylalanine (Pif); 4-aminomethylphenylalanine (Pamfl); 2,4-dichlorophenylalanine (Opef); 3,4-dichlorophenylalanine (Mpcf); 2,4-difluorophenylalanine (Opff); 3,4-difluorophenylalanine (Mpft); pyrid-2-ylalanine (2pAla); pyrid-3-ylalanine (3pAla); pyrid-4-ylalanine (4pAlsa); naphth-1-ylalanine (1nAla); naphth-2-ylalanine (2nAla); thiazolylalanine (taAla); benzothienylalanine (bAla); thienylalanine (tAla); furylalanine (fAla); homophenylalanine (hPhe); homotyrosine (hTyr); homotryptophan (hTrp); pentafluorophenylalanine (5ff); styrylkalanine (sAla); authrylalanine (aAla); 3,3-diphenylalanine (Dfa); 3-amino-5-phenypentanoic acid (Afp); penicillamine (Pen); 1,2,3,4-tetrahydroisoquinoline-3-carboxylic acid (Tic); β-2-thienylalanine (Thi); methionine sulfoxide (Mso); N(w)-nitroarginine (nArg); homolysine (hLys); phosphonomethylphenylalanine (pmPhe); phosphoserine (pSer); phosphothreonine (pThr); homoaspartic acid (hAsp); homoglutanic acid (hGlu); 1-aminocyclopent-(2 or 3)-ene-4 carboxylic acid; pipecolic acid (PA), azetidine-3-carboxylic acid (ACA); 1-aminocyclopentane-3-carboxylic acid; allylglycine (aGly); propargylglycine (pgGly); homoalanine (hAla); norvaline (nVal); homoleucine (hLeu), homovaline (hVal); homoisoleucine (hule); homoarginine (hArg); N-acetyl lysine (AcLys); 2,4-diaminobutyric acid (Dbu); 2,3-diaminobutyric acid (Dab); N-methylvaline (MeVal); hornocysteine (hCys); homoserine (hSer); hydroxyproline (Hyp) and homoproline (hPro). Additional non-encoded amino acids of which the polypeptides described herein may be comprised will be apparent to those of skill in the art (See e.g., the various amino acids provided in Fasman, CRC Practical Handbook of Biochemistry and Molecular Biology, CRC Press, Boca Raton, Fla., pp. 3-70 [1989], and the references cited therein, all of which are incorporated by reference). These amino acids may be in either the L- or D-configuration.

Those of skill in the art will recognize that amino acids or residues bearing side chain protecting groups may also comprise the polypeptides described herein. Non-limiting examples of such protected amino acids, which in this case belong to the aromatic category, include (protecting groups listed in parentheses), but are not limited to: Arg(tos), Cys(methylbenzyl), Cys (nitropyridinesulfenyl), Glu(δ-benzylester), Gln(xanthyl), Asn(N-δ-xanthyl), His(bom), His(benzyl), His(tos), Lys(fmoc), Lys(tos), Ser(O-benzyl), Thr (O-benzyl) and Tyr(O-benzyl).

Non-encoding amino acids that are confonnationally constrained of which the polypeptides described herein may be composed include, but are not limited to, N-methyl amino acids (L-configuration); 1-aminocyclopent-(2 or 3)-ene-4-carboxylic acid; pipecolic acid; azetidine-3-carboxylic acid; homoproline (hPro); and L-aminocyclopentane-3-carboxylic acid.

In some embodiments, the engineered polypeptides are in various forms, for example, such as an isolated preparation, as a substantially purified enzyme, whole cells transformed with gene(s) encoding the enzyme, and/or as cell extracts and/or lysates of such cells. The enzymes can be lyophilized, spray-dried, precipitated or be in the form of a crude paste, as further discussed below.

In some embodiments, the engineered polypeptides are provided on a solid support, such as a membrane, resin, solid carrier, or other solid phase material. A solid support can be composed of organic polymers such as polystyrene, polyethylene, polypropylene, polyfluoroethylene, polyethyleneoxy, and polyacrylamide, as well as co-polymers and grafts thereof. A solid support can also be inorganic, such as glass, silica, controlled pore glass (CPG), reverse phase silica or metal, such as gold or platinum. The configuration of a solid support can be in the form of beads, spheres, particles, granules, a gel, a membrane or a surface. Surfaces can be planar, substantially planar, or non-planar. Solid supports can be porous or non-porous, and can have swelling or non-swelling characteristics. A solid support can be configured in the form of a well, depression, or other container, vessel, feature, or location.

In some embodiments, the engineered polypeptides having glycosyltransferase activity of the present invention can be immobilized on a solid support such that they retain their improved activity, stereoselectivity, and/or other improved properties relative to the reference polypeptide of SEQ ID NO: 2, 4, 6, and/or 8. In such embodiments, the immobilized polypeptides can facilitate the biocatalytic conversion of the substrate compounds or other suitable substrates to the product and after the reaction is complete are easily retained (eg., by retaining beads on which polypeptide is immobilized) and then reused or recycled in subsequent reactions. Such immobilized enzyme processes allow for further efficiency and cost reduction. Accordingly, it is further contemplated that any of the methods of using the glycosyltransferase polypeptides of the present invention can be carried out using the same glycosyltransferase polypeptides bound or immobilized on a solid support.

Methods of enzyme immobilization are well-known in the art. The engineered polypeptides can be bound non-covalently or covalently. Various methods for conjugation and immobilization of enzymes to solid supports (e.g., resins, membranes, beads, glass, etc.) are well known in the art (See e.g, Yi et al., Proc. Biochem., 42(5): 895-898 [2007]; Martin et al., Appl. Microbiol. Biotechnol., 76(4): 843-851 [2007]; Koszelewski et al., J. Mol. Cat. B: Enzymatic, 63: 39-44 [2010]; Truppo et al., Org. Proc. Res. Dev., published online: dx.doi.org/10.1021/op200157c; Hermnanson, Bioconjugate Techniques, 2nd ed., Academic Press, Cambridge, Mass. [2008]; Mateo et al., Biotechnol. Prog., 18(3):629-34 [2002]; and “Bioconjugation Protocols: Strategies and Methods,” In Methods in Molecular Biology, Niemeyer (ed.), Humana Press, New York, N.Y. [2004]; the disclosures of each which are incorporated by reference herein). Solid supports useful for immobilizing the engineered glycosyltransferases of the present invention include but are not limited to beads or resins comprising polymethacrylate with epoxide functional groups, polymethacrylate with amino epoxide functional groups, styrene/DVB copolymer or polymnethacrylate with octadecyl functional groups. Exemplary solid supports useful for immobilizing the engineered glycosyltransferase polypeptides of the present invention include, but are not limited to, chitosan beads, Eupergit C, and SEPABEADs (Mitsubishi), including the following different types of SEPABEAD: EC-EP, EC-HFA/S, EXA252, EXE119 and EXE120.

In some embodiments, the polypeptides described herein are provided in the form of kits. The enzymes in the kits may be present individually or as a plurality of enzymes. The kits can further include reagents for carrying out the enzymatic reactions, substrates for assessing the activity of enzymes, as well as reagents for detecting the products. The kits can also include reagent dispensers and instructions for use of the kits.

In some embodiments, the kits of the present invention include arrays comprising a plurality of different glycosyltransferase polypeptides at different addressable position, wherein the different polypeptides are different variants of a reference sequence each having at least one different improved enzyme property. In some embodiments, a plurality of polypeptides immobilized on solid supports are configured on an array at various locations, addressable for robotic delivery of reagents, or by detection methods and/or instruments. The array can be used to test a variety of substrate compounds for conversion by the polypeptides. Such arrays comprising a plurality of engineered polypeptides and methods of their use are known in the art (See e.g., WO2009/008908A2).

### Polynucleotides Encoding Engineered Glycosyltransferases, Expression Vectors and Host Cells

In another aspect, the present invention provides polynucleotides encoding the engineered glycosyltransferase polypeptides described herein. The polynucleotides may be operatively linked to one or more heterologous regulatory sequences that control gene expression to create a recombinant polynucleotide capable of expressing the polypeptide. Expression constructs containing a heterologous polynucleotide encoding the engineered glycosyltransferase are introduced into appropriate host cells to express the corresponding glycosyltransferase polypeptide.

As will be apparent to the skilled artisan, availability of a protein sequence and the knowledge of the codons corresponding to the various amino acids provide a description of all the polynucleotides capable of encoding the subject polypeptides. The degeneracy of the genetic code, where the same amino acids are encoded by alternative or synonymous codons, allows an extremely large number of nucleic acids to be made, all of which encode the improved glycosyltransferase enzymes. Thus, having knowledge of a particular amino acid sequence, those skilled in the art could make any number of different nucleic acids by simply modifying the sequence of one or more codons in a way which does not change the amino acid sequence of the protein. In this regard, the present invention specifically contemplates each and every possible variation of polynucleotides that could be made encoding the polypeptides described herein by selecting combinations based on the possible codon choices, and all such variations are to be considered specifically disclosed for any polypeptide described herein, including the amino acid sequences disclosed in the sequence listing incorporated by reference herein as the even-numbered sequences in SEQ ID NOS: 1-32.

In various embodiments, the codons are preferably selected to fit the host cell in which the protein is being produced. For example, preferred codons used in bacteria are used to express the gene in bacteria; preferred codons used in yeast are used for expression in yeast; and preferred codons used in mamnmals are used for expression in mammalian cells. In some embodiments, all codons need not be replaced to optimize the codon usage of the glycosyltransferases since the natural sequence will comprise preferred codons and because use of preferred codons may not be required for all amino acid residues. Consequently, codon optimized polynucleotides encoding the glycosyltransferase enzymes may contain preferred codons at about 40%, 50%, 60%, 70%, 80%, or greater than 90% of codon positions of the full length coding region.

In some embodiments, the polynucleotide comprises a codon optimized nucleotide sequence encoding the naturally occurring glycosyltransferase polypeptide amino acid sequence, as represented by SEQ ID NO: 2, 4, 6, and/or 8. In some embodiments, the polynucleotide has a nucleic acid sequence comprising at least 80%, 85%, 90%, 91%, 92%, 93%, 94%, 95%, 96%, 97%, 98%, 99% or more identity to the codon optimized nucleic acid sequences encoding the even-numbered sequences in SEQ ID NOS: 1-32. In some embodiments, the polynucleotide has a nucleic acid sequence comprising at least 80% 85% o, 90%, 91%, 92%, 93%, 94%, 95%, 96%, 97%, 98%, 99% or more identity to the codon optimized nucleic acid sequences in the odd-numbered sequences in SEQ ID NOS: 1-32. The codon optimized sequences of the odd-numbered sequences in SEQ ID NOS: 1-32, enhance expression of the encoded, wild-type glycosyltransferase, providing preparations of enzyme capable of the transferase activity described herein. In some embodiments, the codon optimized polynucleotide sequence enhances expression of the glycosyltransferase by at least 1.2 fold, 1.5 fold or 2 fold or greater as compared to the naturally occurring polynucleotide sequence from Stevia rebaudiana, Streptomyces resistomycificus, Streptomyces antibioticus and/or Paenibacillus sp. YM1.

In some embodiments, the polynucleotides are capable of hybridizing under highly stringent conditions to a reference sequence selected from the odd-numbered sequences in SEQ ID NOS: 1-32, or a complement thereof, and encodes a polypeptide having glycosyltransferase activity.

In some embodiments, as described above, the polynucleotide encodes an engineered polypeptide having glycosyltransferase activity with improved properties as compared to SEQ ID NO: 2, 4, 6, and/or 8, where the polypeptide comprises an amino acid sequence having at least 80%, 85%, 86%, 87%, 88%, 89%, 90%, 91%, 92%, 93%, 94%, 95%, 96%, 97%, 98%, 99% or more identity to a reference sequence, and one or more residue differences as compared to a sequence selected from the even-numbered sequences in SEQ ID NOS: 1-32. In some embodiments, the reference amino acid sequence is selected from the even-numbered sequences in SEQ ID NOS: 1-32. In some embodiments, the reference amino acid sequence is SEQ ID NO: 2. In some embodiments, the reference amino acid sequence is SEQ ID NO: 4. In some further embodiments, the reference amino acid sequence is SEQ ID NO: 8.

In some embodiments, the polynucleotide encodes a glycosyltransferase polypeptide capable of the transferase reaction provided herein, with improved properties as compared to SEQ ID NO: 2, 4, 6, and/or 8, wherein the polypeptide comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 2, 4, 6, and/or 8, and one or more residue differences as compared to SEQ ID NO: 2, 4, 6, and/or 8.

In some embodiments, the polynucleotide encodes a glycosyltransferase polypeptide capable of the transferase reactions provided herein, with improved properties as compared to SEQ ID NO: 2, 4, 6, and/or 8, wherein the polypeptide comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 2, 4, 6, and/or 8, and one or more residue differences as compared to SEQ ID NO: 2, 4, 6, and/or 8.

In some embodiments, the polynucleotide encodes a glycosyltransferase polypeptide capable of the transferase reactions provided herein, with improved properties as compared to SEQ ID NO: 2, 4, 6, and/or 8, wherein the polypeptide comprises an amino acid sequence having at least 80%, 85%, 86%, 87%., 88%, 89%, 90%., 91%, 92%, 93%, 94%., 95%, 96%, 97%, 98%, 99% or more sequence identity to reference sequence SEQ ID NO: 2, 4, 6, and/or 8, and one or more residue differences as compared to SEQ ID NO: 2, 4, 6, and/or 8.

In some embodiments, the polynucleotide encoding the engineered glycosyltransferase comprises a polynucleotide sequence selected from the odd-numbered sequences in SEQ ID NOS: 1-32.

In some embodiments, the polynucleotides are capable of hybridizing under highly stringent conditions to a reference polynucleotide sequence selected from the odd-numbered sequences in SEQ ID NOS: 1-32, or a complement thereof, and encodes a polypeptide having glycosyltransferase activity with one or more of the improved properties described herein. In some embodiments, the polynucleotide capable of hybridizing under highly stringent conditions encodes a glycosyltransferase polypeptide comprising an amino acid sequence having at least 80%, 85%, 86%, 87%, 88%, 89%, 90%, 91%, 92%, 93%, 94%, 95%, 96%, 97%, 98%, 99% or more identity to SEQ ID NO: 2, 4, 6, and/or 8, that has an amino acid sequence comprising one or more residue differences as compared to SEQ ID NO: 2, 4, 6, and/or 8.

In some embodiments, an isolated polynucleotide encoding any of the engineered glycosyltransferase polypeptides provided herein is manipulated in a variety of ways to provide for expression of the polypeptide. In some embodiments, the polyisucleotides encoding the polypeptides are provided as expression vectors where one or more control sequences is present to regulate the expression of the polynucleotides and/or polypeptides. Manipulation of the isolated polynucleotide prior to its insertion into a vector may be desirable or necessary depending on the expression vector. The techniques for modifying polynucleotides and nucleic acid sequences utilizing recombinant DNA methods are well known in the art.

h some embodiments, the control sequences include among other sequences, promoters, leader sequences, polyadenylation sequences, propeptide sequences, signal peptide sequences, and transcription terminators. As known in the art, suitable promoters can be selected based on the host cells used. For bacterial host cells, suitable promoters for directing transcription of the nucleic acid constructs of the present application, include, but are not limited to the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (See e.g., Villa-Kamaroff et al., Proc. Natl Acad. Sci. USA 75: 3727-3731 [1978]), as well as the tac promoter (See e.g., DeBoer et al., Proc. Natl Acad Sci. USA 80: 21-25 [1983]). Exemplary promoters for filamentous fungal host cells, include promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (See e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from any suitable source. In some embodiments, the genes comprise the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other usefil promoters for yeast host cells are known in the art (See e.g., Romanos et al., Yeast 8:423-488 [1992])

In some embodiments, the control sequence is a suitable transcription terminator sequence, a sequence recognized by a host cell to terminate transcription. The terminator sequence is operably linked to the 3′ terminus of the nucleic acid sequence encoding the polypeptide. Any terminator which is fimctional in the host cell of choice finds use in the present invention. For example, exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Various other useful terminators for yeast host cells are known in the art (See e.g., Ronmanos et al., supra).

In some embodiments, the control sequence is a suitable leader sequence, a non-translated region of an mRNA that is important for translation by the host cell. The leader sequence is operably linked to the 5′ terminus of the nucleic acid sequence encoding the polypeptide. Any leader sequence that is functional in the host cell of choice may be used. Exemplary leaders for filamentous fungal host cells are obtained from the genes for Aspergillus oryzae TAKA amylase and Aspergillus nidulans triose phosphate isomerase. Suitable leaders for yeast host cells include, but are not limited to those obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention. Exemplary polyadenylation sequences for filamentous fungal host cells include, but are not limited to those from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are also known in the art (See e.g., Guo and Sherman, Mol. Cell. Bio., 15:5983-5990 [19951]).

In some embodiments, the control sequence is a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to the coding sequence. Any signal peptide coding region that directs the expressed polypeptide into the secretory pathway of a host cell of choice finds use for expression of the engineered glycosyltransferase polypeptides provided herein. Effective signal peptide coding regions for bacterial host cells include, but are not limited to the signal peptide coding regions obtained from the genes for Bacillus NC1B 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are known in the art (See e.g., Simonen and Palva, Microbiol. Rev., 57:109-137 [1993]). Effective signal peptide coding regions for filamentous fingal host cells include, but are not limited to the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells include, but are not limited to those from the genes for Saccharomyces cerevisiae alpha-factor and Saccharomyces cerevisiae invertase.

In some embodiments, the control sequence is a propeptide coding region that codes for an amino acid sequence positioned at the amino terminus of a polypeptide. The resultant polypeptide is referred to as a “proenzyme,” “propolypeptide,” or “zymogen,” in some cases). A propolypeptide can be converted to a mature active polypeptide by catalytic or autocatalytic cleavage of the propeptide from the propolypeptide. The propeptide coding region includes, but is not limited to the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (See e.g., WO 95/33836). Where both signal peptide and propeptide regions are present at the amino terminus of a polypeptide, the propeptide region is positioned next to the arnino terminus of a polypeptide and the signal peptide region is positioned next to the amino terminus of the propeptide region.

In some embodiments, regulatory sequences are also utilized. These sequences facilitate the regulation of the expression of the polypeptide relative to the growth of the host cell. Examples of regulatory systems are those which cause the expression of the gene to be turned on or off in response to a chemical or physical stimulus, including the presence of a regulatory compound. In prokaryotic host cells, suitable regulatory sequences include, but are not limited to the lac, tac, and trp operator systems. In yeast host cells, suitable regulatory systems include, but are not limited to the ADH2 system or GAL1 system. In filamentous fungi, suitable regulatory sequences include, but are not limited to the TAKA alpha-amylase promoter, Aspergillus niger glucoamylase promoter, and Aspergillus oryzae glucoamylase promoter.

In another aspect, the present invention also provides a recombinant expression vector comprising a polynucleotide encoding an engineered glycosyltransferase polypeptide, and one or more expression regulating regions such as a promoter and a terminator, a replication origin, etc., depending on the type of hosts into which they are to be introduced in some embodiments, the various nucleic acid and control sequences described above are joined together to produce a recombinant expression vector which includes one or more convenient restriction sites to allow for insertion or substitution of the nucleic acid sequence encoding the variant glycosyltransferase polypeptide at such sites. Alternatively, the polynucleotide sequence(s) of the present invention are expressed by inserting the polynucleotide sequence or a nucleic acid construct comprising the polynucleotide sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus), that can be conveniently subjected to recombinant DNA procedures and can result in the expression of the variant glycosyltransferase polynucleotide sequence. The choice of the vector will typically depend on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

In some embodiments, the expression vector preferably contains one or more selectable markers, which permit easy selection of transformed cells. A “selectable marker” is a gene the product of which provides for biocide or viral resistance, resistance to heavy metals, prototrophy to auxotrophy, and the like. Examples of bacterial selectable markers include, but are not limited to the dal genes from Bacillus subtilis or Bacillus licheniformis, or markers, which confer antibiotic resistance such as ampicillin, kanamycin, chloramphenicol or tetracycline resistance. Suitable markers for yeast host cells include, but are not limited to ADE2, HIS3, LEU2. LYS2, MET3, TRP1, and URA3. Selectable markers for use in a filamnentous fungal host cell include, but are not limited to, amdS (acetamidase), argB (ornithine carbamoyltransferases), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. In another aspect, the present invention provides a host cell comprising a polynucleotide encoding at least one engineered glycosyltransferase polypeptide of the present application, the polynucleotide being operatively linked to one or more control sequences for expression of the engineered glycosyltransferase enzyme(s) in the host cell. Host cells for use in expressing the polypeptides encoded by the expression vectors of the present invention are well known in the art and include but are not limited to, bacterial cells, such as E. coli, Vibrio fluvialis, Streptomyces and Salmonella typhimurium cells; fungal cells, such as yeast cells (e.g., Saccharomyces cerevisiae and Pichia pastoris [ATCC Accession No. 201178]); insect cells such as Drosophila S2 and Sopodoptera Sf9 cells; animal cells such as CHO, COS, BHK, 293, and Bowes melanoma cells; and plant cells. Exemplary host cells are Escherichia coli strains (e.g., W3110 (ΔfhuA) and BL21).

Accordingly, in another aspect, the present invention provides methods for producing the engineered glycosyltransferase polypeptides, where the methods comprise culturing a host cell capable of expressing a polynucleotide encoding the engineered glycosyltransferase polypeptide under conditions suitable for expression of the polypeptide. In some embodiments, the methods further comprise the steps of isolating and/or purifying the glycosyltransferase polypeptides, as described herein.

Appropriate culture media and growth conditions for the above-described host cells are well known in the art. Polynucleotides for expression of the glycosyltransferase polypeptides may be introduced into cells by various methods known in the art. Techniques include, among others, electroporation, biolistic particle bombardment, liposome mediated transfection, calcium chloride transfection, and protoplast fusion.

The engineered glycosyltransferase with the properties disclosed herein can be obtained by subjecting the polynucleotide encoding the naturally occurring or engineered glycosyltransferase polypeptide to mutagenesis and/or directed evolution methods known in the art, and as described herein. An exemplary directed evolution technique is mutagenesis and/or DNA shuffling (See e.g., Stemmer, Proc. Natl. Acad. Sci. USA 91:10747-10751 [1994]; WO 95/22625; WO 97/0078; WO 97/35966; WO 98/27230; WO 00/42651; WO 01/75767 and U.S. Pat. No. 6,537,746). Other directed evolution procedures that can be used include, among others, staggered extension process (StEP), in vitro recombination (See e.g., Zhao et al., Nat. Biotechnol., 16:258-261 [1998]), mutagenic PCR (See e.g., Caldwell et al., PCR Methods Appl., 3:S136-S140 [1994]1), and cassette mutagenesis (See e.g., Black et al., Proc. Natl. Acad. Sci. USA 93:3525-3529 [1996]).

For example, mutagenesis and directed evolution methods can be readily applied to polynucleotides to generate variant libraries that can be expressed, screened, and assayed. Mutagenesis and directed evolution methods are well known in the art (See e.g., U.S. Pat. Nos. 5,605,793, 5,830,721, 6,132,970, 6,420,175, 6,277,638, 6,365,408, 6,602,986, 7,288,375, 6,287,861, 6,297,053, 6,576,467, 6,444,468, 5,811238, 6,117,679, 6,165,793, 6,180,406, 6,291,242, 6,995,017, 6,395,547, 6,506,602, 6,519,065, 6,506,603, 6,413,774, 6,573,098, 6,323,030, 6,344,356, 6,372,497, 7,868,138, 5,834,252, 5,928,905, 6,489,146, 6,096,548, 6,387,702, 6,391,552, 6,358,742, 6,482,647, 6,335,160, 6,653,072, 6,355,484, 6,303,344, 6,319,713, 6,613,514, 6,455,253, 6,579,678, 6,586,182, 6,406,855, 6,946,296, 7,534,564, 7,776,598, 5,837,458, 6,391,640, 6,309,883, 7,105,297, 7,795,030, 6,326,204, 6,251,674, 6,716,631, 6,528,311, 6,287,862, 6,335,198, 6,352,859, 6,379,964, 7,148,054, 7,629,170, 7,620,500, 6,365,377, 6,358,740, 6,406,910, 6,413,745, 6,436,675, 6,961,664, 6,537,746, 7,430,477, 7,873,499, 7,702,464, 7,783,428, 7,747,391, 7,747,393, 7,751,986, 6,376,246, 6,426,224, 6,423,542, 6,479,652, 6,319,714, 6,521,453, 6,368,861, 7,421,347, 7,058,515, 7,024,312, 7,620,502, 7,853,410, 7,957,912, 7,904,249, 8,383,346, 8,504,498, 8,768,871, 8,762,066, 8,849,575, and all related non-US counterparts; Ling et al., Anal. Biochem., 254:157-78 [1 997]; Dale et al., Meth. Mol. Biol., 57:369-74 [1996]; Smith, Ann. Rev. Genet., 19:423-462 [1985]; Botstein et al., Science, 229:1193-1201 [1985]; Carter, Biochem. J., 237:1-7 [1986]; Kramer et al., Cell, 38:879-887 [1984]; Wells et al., Gene, 34:315-323 [1985]; Minshull et al., Curr. Op. Chem. Biol., 3:284-290 [1999]; Christians et al., Nat. Biotechnol., 17:259-264 [1999]; Crameri et al., Nature, 391:288-291 [1998]; Crarneri, et al., Nat. Biotechnol., 15:436-438 [1997]; Zhang et al., Proc. Nat. Acad. Sci. U.S.A., 94:4504-4509 [1997]; Crarneri et al., Nat. Biotechnol., 14:315-319 [1996]; Stemmer, Nature, 370:389-391 [1994]; Stemmer, Proc. Nat. Acad. Sci. USA, 91:10747-10751 [1994]; WO 95/22625; WO 97/0078; WO 97/35966; WO 98/27230; WO 00/42651; WO 01/75767; WO 2009/152336, WO 2009/102901, WO 2009/102899, WO 2011/035105, WO 2013/138339, WO 2013/003290, WO 2014/120819, WO 2014/120821, WO 2015/0134315, and WO 2015/048573, all of which are incorporated herein by reference).

In some embodiments, the enzyme clones obtained following mutagenesis treatment are screened by subjecting the enzymes to a defined temperature (or other assay conditions, such as testing the enzyme's activity over a broad range of substrates) and measuring the amount of enzyme activity remaining after heat treatments or other assay conditions. Clones containing a polynucleotide encoding a glycosyltransferase polypeptide are then sequenced to identify the nucleotide sequence changes (if any), and used to express the enzyme in a host cell. Measuring enzyme activity from the expression libraries can be performed using any suitable method known in the art (e.g., standard biochemistry techniques, such as HPLC analysis).

In some embodiments, the clones obtained following mutagenesis treatment can be screened for engineered glycosyltransferases having one or more desired improved enzyme properties (e.g., improved transferase activity). Measuring enzyme activity from the expression libraries can be performed using the standard biochemistry techniques, such as HPLC analysis, LC-MS/MS analysis, and/or derivatization of products (pre or post separation), as known in the art (e.g., using dansyl chloride or OPA; See e.g., Yaegaki et al., J Chromatogr. 356(1):163-70 [1986]).

For engineered polypeptides of known sequence, the polynucleotides encoding the enzyme can be prepared by standard solid-phase methods, according to known synthetic methods. In some embodiments, fragments of up to about 100 bases can be individually synthesized, then joined (e.g., by enzymatic or chemical ligation methods, or polymerase mediated methods) to form any desired continuous sequence. For example, polynucleotides and oligonucleotides disclosed herein can be prepared by chemical synthesis using the classical phosphoramidite method (See e.g., Beaucage et al., Tetra. Le., 22:1859-69 [1981]; and Matthes et al., EMBO J., 3:801-05 [1984]), as it is typically practiced in automated synthetic methods. According to the phosphoramidite method, oligonucleotides are synthesized (e.g., in an automatic DNA synthesizer), purified, annealed, ligated and cloned in appropriate vectors.

Where the sequence of the engineered polypeptide is known, the polynucleotides encoding the enzyme can be prepared by standard solid-phase methods, according to known synthetic methods. In some embodiments, fragments of up to about 100 bases can be individually synthesized, then joined (e.g., by enzymatic or chemical ligation methods, or polymerase mediated methods) to fomrm any desired continuous sequence. For example, polynucleotides and oligonucleotides encoding portions of the glycosyltransferase can be prepared by chemical synthesis as known in the art (e.g., the classical phosphoramidite method of Beaucage et al., Tet. Lett. 22:1859-69 [1981], or the method described by Matthes et al., EMBO J. 3:801-05 [1984]) as typically practiced in automated synthetic methods. According to the phosphoramidite method, oligonucleotides are synthesized (e.g., in an automatic DNA synthesizer), purified, annealed, ligated and cloned in appropriate vectors. In addition, essentially any nucleic acid can be obtained from any of a variety of commercial sources. In some embodiments, additional variations can be created by synthesizing oligonucleotides containing deletions, insertions, and/or substitutions, and combining the oligonucleotides in various permutations to create engineered glycosyltransferases with improved properties.

Accordingly, in some embodiments, a method for preparing the engineered glycosyltransferases polypeptide comprises: (a) synthesizing a polynucleotide encoding a polypeptide comprising an amino acid sequence having at least about 80%, 85%, 86%, 87%, 88%, 89%, 90%, 91%, 92%, 93%, 94%, 95%, 96%, 97%, 98%, or 99% or more sequence identity to an amino acid sequence selected from SEQ ID NO: 2, 4, 6, and/or 8, and having one or more residue differences as compared to SEQ ID NO: 2, 4, 6, and/or 8; and (b) expressing the glycosyltransferase polypeptide encoded by the polynucleotide.

Accordingly, in some embodiments, a method for preparing the engineered glycosyltransferases polypeptide comprises: (a) synthesizing a polynucleotide encoding a polypeptide comprising an amino acid sequence having at least about 80%, 85%, 86%, 87%, 88%, 89%, 90%, 91%, 92%, 93%, 94%, 95%, 96%, 97%, 98%, or 99% or more sequence identity to an amino acid sequence selected from SEQ ID NO: 2, 4, 6, and/or 8, and having one or more residue differences as compared to SEQ ID NO: 2, 4, 6, and/or 8; and (b) expressing the glycosyltransferase polypeptide encoded by the polynucleotide.

In some embodiments of the method, the polynucleotide encodes an engineered glycosyltransferase that has optionally one or several (e.g., up to 3, 4, 5, or up to 10) amino acid residue deletions, insertions and/or substitutions. In some embodiments, the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 18, 20, 21, 22, 23, 24, or 25 amino acid residue deletions, insertions and/or substitutions. In some embodiments, the substitutions can be conservative or non-conservative substitutions.

In some embodiments, any of the engineered glycosyltransferase enzymes expressed in a host cell can be recovered from the cells and/or the culture medium using any one or more of the well known techniques for protein purification, including, among others, lysozyme treatment, sonication, filtration, salting-out, ultra-centrifugation, and chromatography. Suitable solutions for lysing and the high efficiency extraction of proteins from bacteria, such as E. coli, are commercially available (e.g., CelLytic BTM, Sigma-Aldrich, St. Louis Mo.).

Chromatographic techniques for isolation of the glycosyltransferase polypeptide include, among others, reverse phase chromatography high performance liquid chromatography, ion exchange chromatography, gel electrophoresis, and affinity chromatography. Conditions for purifying a particular enzyme will depend, in part, on factors such as net charge, hydrophobicity, hydrophilicity, molecular weight, molecular shape, etc., and will be apparent to those having skill in the art.

In some embodiments, affinity techniques may be used to isolate the improved glycosyltransferase enzymes. For affinity chromatography purification, any antibody which specifically binds the glycosyltransferase polypeptide may be used. For the production of antibodies, various host animals, including but not limited to rabbits, mice, rats, etc., may be immunized by injection with a glycosyltransferase polypeptide, or a fragment thereof. The glycosyltransferase polypeptide or fragment may be attached to a suitable carrier, such as BSA, by means of a side chain functional group or linkers attached to a side chain functional group. In some embodiments, the affinity purification can use a specific ligand bound by the glycosyltransferase, such as poly(L-proline) or dye affinity column (See e.g., EP0641862; Stellwagen, “Dye Affinity Chromatography,” In Current Protocols in Protein Science, Unit 9.2-9.2.16 [2001]).

### Methods for Using Glycosyltransferases to β-Glycosylate Compounds of Interest

In some embodiments, the methods, processes, and systems provided herein facilitate the conversion of a substrate to a β-glycosylated product. In some embodiments, the substrate stevioside is converted to the β-glucosylated product rebaudioside A. In some embodiments, this glycosylation reaction is catalyzed by an enzyme. In some embodiments, the enzyme is a glycosyltransferase, while in some alternative embodiments the enzyme is a phosphorylase. In some further embodiments, the glycosyltransferase or phosphorylase uses glucose-1-phosphate (e.g., α-glucose-1-phosphate), as a glucosyl donor. In some additional embodiments, certain moieties of the substrate (e.g., hydroxyl groups), act as a glycosyl (e.g., glucosyl) acceptor. Some non-limiting examples of glycosyltransferases that find use in the present invention include promiscuous bacterial UDP-glucose-dependent glycosyltransferases (e.g., the glycosyltransferase of SEQ NO: 4, 6, or 12), and phosphorylases that possess β-glycosyl cleavage activity to produce α-glucose-1-phosphate (e.g., a laminaribiose phosphorylase such as that of SEQ NO: 8 or 14). In some embodiments, the enzyme contacts the substrate in vitro, while in some alternative embodiments, the enzyme contacts the substrate in vivo. In still some additional embodiments, the substrate and enzyme are produced and contacted within an engineered host cell.

The suitable reaction conditions under which the polypeptides carry out the conversion can be determined by those of skill in the art. The Examples provide exemplary reaction conditions, including the concentrations or amounts of polypeptide, substrate, co-substrate (e.g., glucose-1-phosphate), buffer, co-solvent, pH, temperature, and reaction time. For example, in some embodiments, reactions are performed with substrate concentrations up to 5 mM or the solubility limit of the substrate, up to 5 mM cosubstrate, or the solubility limit of the cosubstrate, in 25-100 mM buffer, with 0-20% ethanol, at pH 5-8, at 30-65° C., for 5 m-18 h. In some embodiments, reactions may be additionally performed with a co-enzyme and second co-substrate in order to regenerate glucose-1-phosphate in situ, which is useful for perforning the reaction in a more inexpensive manner. For example, the co-enzyme may be a retaining phosphorylase, such as sucrose phosphorylase (eg., even numbered SEQ NO: 16-32), and the additional co-substrate may be the appropriate α-linked disaccharide, trisaccharide, or oligosaccharide enzyme substrate, such as sucrose. The co-enzyme may be an inverting phosphorylase, such as cellobiose phosphorylase (E.C. 2.4.1.20), and the additional co-substrate may be the appropriate β-linked disaccharide, trisaccharide, or oligosaccharide enzyme substrate, such as cellobiose. However, it is not intended that the present invention be limited to the specific reactions and conditions as set forth in the Examples. Various features and embodiments of the invention are illustrated in the following representative examples, which are intended to be illustrative, and not limiting.

### EXPERIMENTAL

The following Examples, including experiments and results achieved, are provided for illustrative purposes only and are not to be construed as limiting the present invention.

In the experimental disclosure below, the following abbreviations apply: M (molar); mM (millimolar), uM and μM (micromolar); nM (nanomolar); mol (moles); gm and g (gram); mg (milligrams); ug and μg (micrograms): L and l (liter); ml and mL (milliliter); cm (centimeters); min (millimeters); um and μm (micrometers); sec. (seconds); min(s) (minute(s)); h(s) and hr(s) (hour(s)); U (units); MW (molecular weight); rpm (rotations per minute); psi and PSI (pounds per square inch); ° C. (degrees Centigrade); RT and rt (room temperature); CAM and carn (chloramphenicol); PMBS (polynyxin B sulfate); IPTG (isopropyl β-D-1-thiogalactopyranoside); UGT (uridine 5′-diphosphoglucose glycosyltransferase); LB (Luria broth); TB (terrific broth): SFP (shake flask powder); CDS (coding sequence); DNA (deoxyribonucleic acid); RNA (ribonucleic acid); E. coli W3110 (commonly used laboratory E. coli strain, available from the Coli Genetic Stock Center [CGSC], New Haven, Conn.); HTP (high throughput); HPLC (high performance liquid chromatography); MS (mass spectrometry); FIOPC (fold improvements over positive control); Sigma-Aldrich (Sigma-Aldrich, St. Louis, Mo.; Difco (Difco Laboratories, BD Diagnostic Systems, Detroit, Mich.); Microfluidics (Microfluidics, Westwood, Mass.); ChromaDex (ChromaDex, Inc., Irvine, Calif.); and Thermotron (Thermotron, Holland, Mich.).

### Example 1

### Synthesis, Optimization, and Assaying of UGT Enzymes with Glucosylation Activity

In this Example, methods used in the synthesis, optimization and assaying of UGT enzymes with glucosylation activity are described.

### Gene Synthesis and Optimization:

The polynucleotide sequence (SEQ ID NO: 1) encoding the wild-type Stevia rebaudiana polypeptide (SEQ ID NO: 2) reported to glucosylate steviolbioside to rebaudioside B and glucosylate stevioside to rebaudioside A, was codon-optimized and synthesized as the gene of SEQ ID NO: 9. The polynucleotide sequence (SEQ ID NO: 3) encoding the wild-type Streptomyces resistomycificus glycosyltransferase polypeptide (SEQ ID NO: 4), is a homolog (with 82% sequence identity) of the wild-type Streptomyces antibioticus oleandomycin glycosyltrausferase (SEQ ID NO:5) sequence reported to glucosylate oleandomycin and have promiscuous activity with other nucleotide sugar donors and toward other substrates, was similarly codon-optimized and synthesized (SEQ ID NO: 11). These synthetic genes (SEQ ID NOS: 9 and 11) were individually cloned into a pCK110900 vector system (See e.g., US Pat. Appln. Publn. No. 2006/0195947, which is hereby incorporated by reference herein) and subsequently expressed in E. coli W3110 (ΔfhuA). The E. coli strain W3110 expressed the UGT enzymes under the control of the lac promoter.

### Production of Shake Flask Powders (SFP):

A shake-flask procedure was used to generate the glycosyltransferase polypeptide shake flask powders (SFP) for characterization assays used in the biocatalytic processes described herein. Shake flask powder (SFP) preparation of enzymes provides a more purified preparation (e.g., up to >30% of total protein) of the enzyme as compared to the cell lysate used in HTP assays and also allows for the use of more concentrated enzyme solutions. A single colony of E. coli containing a plasmid encoding an engineered polypeptide of interest was inoculated into 5 mL Luria Bertani broth containing 30 μg/ml chloramphenicol and 1% glucose. Cells were grown overnight (at least 16 hours) in an incubator at 30° C. with shaking at 250 ipm. The culture was diluted into 250 mL Terrific Broth (12 g/L bacto-tryptone, 24 g/L yeast extract, 4 mL/L glycerol, 65 mM potassium phosphate, pH 7.0, 1 mM MgSO₄) containing 30 μg/ml CAM, in a I L flask to an optical density of 600 nm (OD600) of 0.2 and allowed to grow at 30° C. Expression of the glycosyltransferase gene was induced by addition of IPTG to a final concentration of I mM when the OD600 of the culture was 0.6 to 0.8. Incubation was then continued overnight (at least 16 hours). Cells were harvested by centrifugation (5000 rpm, 15 min, 4° C.) and the supernatant discarded. The cell pellet was resuspended in two volumes of 25 mM triethanolamine buffer, pH 7.5, and passed through a MICROFLUIDIZER high pressure homogenizer (Microfluidics), with standard E. coli lysis settings and maintained at 4° C. Cell debris was removed by centrifugation (10,000 rpm, 45 minutes, 4° C.). The cleared lysate supernatant was collected and frozen at −80° C. and then lyophilized to produce a dry shake-flask powder of crude polypeptide.

### Assay of SFP for Stevioside Glycosylation:

SFP was reconstituted to provide 20 g/L powder. Then, 50 μL of these stocks were diluted in 200 μL total reaction volume of 50 mM Tris-HCl buffer, pH 7.5, with 3 mM MgSO4 and 1 mM stevioside (ChromaDex, >94% purity), with or without 5 mM α-glucose-1-phosphate. The reaction was performed at 30° C. in a Thermotron titre-plate shaker with 300 RPM shaking for 16-18 h.

### HPLC-MS/MS Analysis:

The reaction described above was quenched with 0.5 volume/volume acetonitrile with 2% formic acid and precipitated by centrifugation. Glycosylated stevioside products were detected in the supernatant by LC-MS/MS with the following instrument and parameters:

| Instrument | Agilent HPLC 1200 series, Sciex 4000 QTrap | |
|---|---|---|
| Column | Poroshell 120 EC C18 50 x 3.0 mm, 2.7 μm with | |
| | Poroshell 120 EC C18 5 x 3.0 mm, 2.7 μm guard | |
| | column (Agilent Technologies) | |
| Mobile phase | Gradient (A: 0.1% formic acid in water; B: 0.1% | |
| | formic acid in methanol) | |
| | Time (m) | % B |
| | 0 | 60 |
| | 0.50 | 60 |
| | 1.00 | 70 |
| | 4.33 | 70 |
| | 5.00 | 95 |
| | 5.33 | 95 |
| | 5.34 | 60 |
| | 6.00 | 60 |

| | |
|---|---|
| Flow rate | 0.8 mL/m |
| Run time | 6 m |
| Peak retention times | Rebaudioside A: 2.35 m; Product 176: 1.76 m, |
| | Product 218: 2.18 m, Product 222: 2.22 in. |
| Column temperature | 40° C. |
| Injection volume | 10 μL |
| MS detection | Sciex 4000 QTrap; MRM 990/828 (for steviol |
| | tetraglycosides, e.g., rebaudioside A), 1152/828 |
| | (for steviol pentaglycosides, e.g., rebaudioside |
| | D), 1314/828 (steviol hexaglycosides, e.g., |
| | rebaudioside M), 828/666 (for steviol |
| | triglycosides, e.g., stevioside), 666/504 (steviol |
| | diglycosides, e.g., rubusoside) |
| MS conditions | MODE: MRM; CUR: 30; IS: 4750; CAD: high; |
| | TEM: 550° C.; GSI: 50; GS2: 50; DP: 150; EP: |
| | 10; CXP: 14; DT: 50 ms for each transition |
| | For the first three transitions: CE: 85For the last |
| | two transitions: CE: 60 |

Novel activity was detected for the polypeptides encoded by SEQ ID NO: 9 and 11. For the glucosyltransferase polypeptide encoded by SEQ ID NO: 9, two products were detected, one co-eluting with rebaudioside A and one eluting at 2.22 m. These products were detected in the presence of α-glucose-1-phosphate, but not in the absence of α-glucose-1-phosphate. These products were not present in a negative control sample. For the giucosyitransferase polypeptide encoded by SEQ ID NO: 11, two products were detected at retention times 1.76 and 2.18 m. These products were detected in the presence of α-glucose-1-posphate and at reduced levels in the absence of α-glucose-1-phosphate. These products were not present in a negative control sample. Thus, the present invention provides a novel process for glucosylating steviol glycoside substrates. In addition, the present invention provides the first enzymes in this class, including the wild-type enzymes encoded by SEQ ID NO: 2 and 4, to be used with α-glucose-1-phosphate instead of uridine 5′-diphosphoglucose to glycosylate steviol substrates.

### Example 2

### In Situ Formation of Glucose-1-Phosphate

In this Example experiments to assess the in situ formation of glucose-1-phosphate for UDP-glucose-independent glucosylation of substrates (See, FIG. 3) are described. Gene synthesis and optimization, as well as production of shake flask powders are performed as described in Example 1.

### Assay of SFP:

SFP is reconstituted to 20 g/L powder. Then, 20 μL of SFP from E. coli expressing SEQ ID NO: 9 or 11 and 10 μL of SFP from E. coli expressing odd-numbered SEQ ID NO: 15-31 or another disaccharide phosphorylase or a negative control are diluted in 200 μL total reaction volume of 50 mM Tris-HCl buffer, pH 7.5, with 3 mM MgSO₄, 0.3 M sucrose (or the disaccharide corresponding to the disaccharide phosphorylase), 5 mM inorganic phosphate, and 1 mM stevioside (ChromaDex, >94% purity), with 0-5 mM α-glucose-1-phosphate. The reaction is performed at 30° C. in a Thermotron titre-plate shaker with 300 RPM shaking for 16-18 h.

### HPLC-MS/MS Analysis:

The reaction is quenched and analyzed by LC-MS/MS as described in Example 1. In situ formation of α-glucose-1-phosphate is demonstrated by increased conversion of stevioside to glucosylated products at retention times 1.76, 2.18, 2.22, and 2.35 m in the presence of SFP from E. coli expressing odd-numbered SEQ NO: 15-31 or another disaccharide phosphorylase relative to the same samples in the presence of SFP from E. coli expressing a negative control.

### Example 3

### Evolution and Screening of Variants

This Example describes experiments conducted during the evolution and screening of engineered polypeptides derived from SEQ ID NO: 9 and 11 for improved substrate glucosylation using α-glucose-1-phosphate.

Directed evolution begins with the polynucleotides of SEQ ID NO: 9 or 11, which encode the polypeptide of SEQ ID NO: 10 or 12, respectively, as the starting “backbone” gene sequence. Libraries of engineered polypeptides are generated using various well-known techniques (e.g., saturation mutagenesis, recombination of previously identified beneficial amino acid differences) and screened using HTP assay and analysis methods that measure the ability of the engineered polypeptides to carry out glucosylation of the desired substrate using glucose-1-phosphate or a recycling system for glucose-1-phosphate consisting of an inexpensive disaccharide, inorganic phosphate, and a wild-type or engineered disaccharide phosphorylase, as described in Examples 1 and 2.

After screening, the engineered polypeptides showing the most improvement over the starting backbone sequence are used as backbone sequences for the construction of further libraries, and the screening process repeated to evolve the polypeptides for the desired activity. Due to the promiscuity of the enzyme for various substrates, this process can be repeated to develop biocatalysts that use an inexpensive co-substrate for the glucosylation of many substrates of interest.

### Example 4

### Synthesis, Optimization, and Assaying Phosphorylase Enzymes with Glucosylation Activity

In this Example, experiments conducted to synthesize, optimize and assay phosphorylase enzymes having glucosylation activity are described.

### Gene Synthesis and Optimization:

The polynucleotide sequence (SEQ ID NO: 7) encoding the wild-type Paenibacillus sp. YM1 laminaribiose phosphorylase polypeptide (SEQ ID NO: 8), reported to phosphorylyse laminaribiose to release glucose and form α-glucose-1-phosphate, was codon-optimized and synthesized as the gene of SEQ ID NO: 13. The synthetic gene of SEQ ID NO: 13 was cloned into a pCK110900 vector system (See e.g., US Pat. Appln. Publn. No. 20060195947, which is hereby incorporated by reference herein) and subsequently expressed in E. coli W3110 (ΔfhuA). The E. coli strain W3110 expressed the enzyme under the control of the lac promoter.

### Production of High-Throughput (HTIP) Lysates:

E. coli cells expressing the polypeptide genes of interest were grown and induced in 96-well plates, pelleted, lysed in 250 μL lysis buffer (0.5 g/L lysozyme and 0.5 g/L PMBS in 20 mM Tris-HCi buffer, pH 7.5) with low-speed shaking for 2 h on a titre-plate shaker at room temperature. The plates were then centrifuged at 4000 rpm and 4° C. for 20 m and the cleared lysate supernatant was used in the assay reactions described herein.

### Assay for Glucosylation of Stevioside:

In this assay, 50 μL cleared lysates were diluted in 200 μL total reaction volume of 50 mM sodium acetate buffer, pH 5.5, with 1.65 mM α-glucose-1-phosphate and 0.5 mM stevioside (ChromaDex, >94% purity). The reaction was performed at 40° C. in a Thennotron titre-plate shaker with 300 RPM shaking for 18 h.

### HPLC-MS/MS Analysis:

The reaction was quenched and analyzed by LC-MS/MS as described in Example L. A glucosylated stevioside product with retention time 2.18 m was observed in the presence of the laminaribiose phosphorylase but not for the negative control sample. This result indicates that a laminaribiose phosphorylase may be used with α-glucose-1-phosphate as a novel method for α-glucosidation of stevioside and other substrates. As described in Example 3, α-glucose-1-phosphate can be recycled using an inexpensive disaccharide, inorganic phosphate, and a disaccharide phosphorylase.

1. A method for glycosylation of a substrate to produce a beta-glycosylated product, comprising the steps of: providing at least one glycosyl group donor, a least one glycosyl group acceptor, and at least one glycosyltransferase enzyme; contacting the glycosyl group donor and glycosyl group acceptor with the glycosyltransferase enzyme under conditions such that the glycosyl group acceptor is glycosylated to produce at least one product having beta-glucose linkages.

2. The method of claim 1, wherein said glycosyl group donor is a glycosylphosphate.

3. The method of claim 2, wherein said glycosyl group donor is glucose-1-phosphate.

4. The method of claim 1, wherein said glycosyl group acceptor is selected from glycosyl, alkoxy, carboxy, aminocarbonyl, heteroalkyl, heteroalkenyl, heteroalkynyl, carboxyalkyl, aminoalkyl, haloalkyl, alkylthioalkyl, heterocycloalkyl, heteroaryl, and heteroarylalkyl groups.

5. The method of claim 1, wherein said product having beta-glucose linkages is a steviol glycoside.

6. The method of claim 1, wherein said glycosyl group acceptor is stevioside, said glycosyl group donor is alpha-glucose-1-phosphate, and said product having beta-glucose linkages is rebaudioside A.

7. The method of claim 1, wherein said glycosyltransferase is selected from the polypeptides set forth in SEQ ID NOS:2, 4, 6, 8, 10, 12, and 14.

8. A method for production of glucose-1-phosphate, comprising the steps of: providing a phosphorylase, inorganic phosphate, and a disaccharide, trisaccharide, or oligosaccharide substrate of the phosphorylase; contacting said phosphorylase, inorganic phosphate, and saccharide under conditions such that said saccharide is cleaved to produce a monosaccharide and glucose-1-phosphate.

9. (canceled)

10. The method of claim 9, wherein said glucosyl group acceptor is selected from glycosyl, alkoxy, carboxys, aminocarbonyl, heteroalkyl, heteroalkenyl, heteroalkynyl, carboxyalkyl, aminoalkyl, haloalkyl, alkylthioalkyl, heterocycloalkyl, heteroaryl, and heteroarylalkyl groups.

11. The method of claim 9, wherein said phosphorylase is sucrose phosphorylase, said saccharide is sucrose, said monosaccharide produced is sucrose, and said glucose-1-phosphate produced is α-glucose-1-phosphate.

12. The method of claim 8, wherein said phosphorylase comprises a polypeptide sequence selected from SEQ ID NOS:16, 18, 20, 22, 24, 26, 28, 30, and 32.


SEQUENCE LISTING (32 sequences)

SEQ ID NO: 1 - DNA, 1326 bp, Artificial Sequence
  Wild type UDP-glycosyltransferase 76G1 from      Stevia
atggaaaataaaacggagaccaccgttcgccggcgccggagaataatattattcccggtaccatttcaaggccacattaacccaattcttcagctagccaatgtgttgtactctaaaggattcagtatcaccatctttcacaccaacttcaacaaacccaaaacatctaattaccctcacttcactttcagattcatcctcgacaacgacccacaagacgaacgcatttccaatctaccgactcatggtccgctcgctggtatgcggattccgattatcaacgaacacggagctgacgaattacgacgcgaactggaactgttgatgttagcttctgaagaagatgaagaggtatcgtgtttaatcacggatgctctttggtacttcgcgcaatctgttgctgacagtcttaacctccgacggcttgttttgatgacaagcagcttgtttaattttcatgcacatgtttcacttcctcagtttgatgagcttggttacctcgatcctgatgacaaaacccgtttggaagaacaagcgagtgggtttcctatgctaaaagtgaaagacatcaagtctgcgtattcgaactggcaaatactcaaagagatattagggaagatgataaaacaaacaagagcatcttcaggagtcatctggaactcatttaaggaactcgaagagtctgagctcgaaactgttatccgtgagatcccggctccaagtttcttgataccactccccaagcatttgacagcctcttccagcagcttactagaccacgatcgaaccgtttttcaatggttagaccaacaaccgccaagttcggtactgtatgttagttttggtagtactagtgaagtggatgagaaagatttcttggaaatagctcgtgggttggttgatagcaagcagtcgtttttatgggtggttcgacctgggtttgtcaagggttcgacgtgggtcgaaccgttgccagatgggttcttgggtgaaagaggacgtattgtgaaatgggttccacagcaagaagtgctagctcatggagcaataggcgcattctggactcatagcggatggaactctacgttggaaagcgtttgtgaaggtgttcctatgattttctcggattttgggctcgatcaaccgttgaatgctagatacatgagtgatgttttgaaggtaggggtgtatttggaaaatgggtgggaaagaggagagatagcaaatgcaataagaagagttatggtggatgaagaaggagaatacattagacagaatgcaagagttttgaaacaaaaggcagatgtttctttgatgaagggtggt

SEQ ID NO: 2 - protein, 442 aa, Artificial Sequence
  Wild type UDP-glycosyltransferase 76G1 from      Stevia
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTRASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGG

SEQ ID NO: 3 - DNA, 1203 bp, Artificial Sequence
  Wild type macrolide glycosyltransferase from      Streptomyces
atgaccagtcgctcgcacatcgccatgttctccatcgccgcccacgggcacgtgaacccgagcctcgaggtgatccgcgagctcgtcgcgcgcgggcaccgggtgacgtacgcgattccgcccgtcttcgccgggaaggtcgccgaggccggcgccgaggtgaagctctggaactccaccctgccctcgcccgacgacgacccacaggcatggggcacgaccctgctggacaacgtggagccgttcctggccgacgcgatccaggcgctcccgcagctgatcgaggcgtacgaaggggacgagcccgacctcgtgctgcacgacatcacctcctacccggcccgcgtcctggcccaccgctggggcgtgcccgcggtctccctgtcgccgaacctcgtcgcctgggagggctacgagaaggaggtggccgagccgatgtgggcggagccgaagcggaccgagcgcgggcgggcctactacgcccgcttccacgcctggctggaggagaacggcgtcaccctgcacccggacgacttcgtgggccgccccgcccgctgtctcgtcctgatcccgaaggcgctgcagcccaacgccgaccgggtggacgagggcgtgtactccttcgtgggcgcctgccagggtgaccgcggcgcggagggggagtggcggcggcccgccggcgccgagaaggtcgccctggtctcgctcgggtcctccttcaccaagcagccggacttctaccgggagtgcgtcagggcgttcggcgagctgccgggctggcacctggtgctccagatcggcaggcacgtcgacccggccgacctgggggacgtaccggagaacgtcgaagtgcgctcctgggtaccccagttggcgatcctcaaggaggccgatctgttcgtcacgcacgccggcgcgggcggcagccaggagggtctggccacggccacgccgatgatcgccgtaccgcaggccgtggaccagttcggcaacgcggacatgctccaggggctcggtgtcgcccgccgcctcgacaccgaggaggcgaccgccgaggccctcagggcggccgccctcgctctcgtcgacgaccccgaagtggcccgtcgggcgcgggacatccaggctcagatggcgggggagggcggcacgcgccgggcggccgacctgatcgaggccgagctgccggtacggcaggcgtag

SEQ ID NO: 4 - protein, 400 aa, Artificial Sequence
  Wild type macrolide glycosyltransferase from      Streptomyces
MTSRSHIAMFSIAAHGHVNPSLEVIRELVARGHRVTYAIPPVFAGKVAEAGAEVKLWNSTLPSPDDDPQAWGTTLLDNVEPFLADAIQALPQLIEAYEGDEPDLVLHDITSYPARVLAHRWGVPAVSLSPNLVAWEGYEKEVAEPMWAEPKRTERGRAYYARFHAWLEENGVTLHPDDFVGRPARCLVLIPKALQPNADRVDEGVYSFVGACQGDRGAEGEWRRPAGAEKVALVSLGSSFTKQPDFYRECVRAFGELPGWHLVLQIGRHVDPADLGDVPENVEVRSWVPQLAILKEADLFVTHAGAGGSQEGLATATPMIAVPQAVDQFGNADMLQGLGVARRLDTEEATAEALRAAALALVDDPEVARRARDIQAQMAGEGGTRRAADLIEAELPVRQA

SEQ ID NO: 5 - DNA, 1248 bp, Artificial Sequence
  Wild type macrolide glycosyltransferase from      Streptomyces
atgaccacccagaccactcccgcccacatcgccatgttctccatcgccgcccacggccatgtgaaccccagcctggaggtgatccgtgaactcgtcgcccgcggccaccgggtcacgtacgccattccgcccgtcttcgccgacaaggtggccgccaccggcgcccggcccgtcctctaccactccaccctgcccggccccgacgccgacccggaggcatggggaagcaccctgctggacaacgtcgaaccgttcctgaacgacgcgatccaggcgctcccgcagctcgccgatgcctacgccgacgacatccccgatctcgtcctgcacgacatcacctcctacccggcccgcgtcctggcccgccgctggggcgtcccggcggtctccctctccccgaacctcgtcgcctggaagggttacgaggaggaggtcgccgagccgatgtggcgcgaaccccggcagaccgagcgcggacgggcctactacgcccggttcgaggcatggctgaaggagaacgggatcaccgagcacccggacacgttcgccagtcatccgccgcgctccctggtgctcatcccgaaggcgctccagccgcacgccgaccgggtggacgaagacgtgtacaccttcgtcggcgcctgccagggagaccgcgccgaggaaggcggctggcagcggcccgccggcgcggagaaggtcgtcctggtgtcgctcggctcggcgttcaccaagcagcccgccttctaccgggagtgcgtgcgcgccttcgggaacctgcccggctggcacctcgtcctccagatcggccggaaggtgacccccgccgaactgggggagctgccggacaacgtggaggtgcacgactgggtgccgcagctcgcgatcctgcgccaggccgatctgttcgtcacccacgcgggcgccggcggcagccaggaggggctggccaccgcgacgcccatgatcgccgtaccgcaggccgtcgaccagttcggcaacgccgacatgctccaagggctcggcgtcgcccggaagctggcgaccgaggaggccaccgccgacctgctccgcgagaccgccctcgctctggtggacgacccggaggtcgcgcgccggctccggcggatccaggcggagatggcccaggagggcggcacccggcgggcggccgacctcatcgaggccgaactgcccgcgcgccacgagcggcaggagccggtgggcgaccgacccaacggtgggtga

SEQ ID NO: 6 - protein, 415 aa, Artificial Sequence
  Wild type macrolide glycosyltransferase from      Streptomyces
MTTQTTPAHIAMFSIAAHGHVNPSLEVIRELVARGHRVTYAIPPVFADKVAATGARPVLYHSTLPGPDADPEAWGSTLLDNVEPFLNDAIQALPQLADAYADDIPDLVLHDITSYPARVLARRWGVPAVSLSPNLVAWKGYEEEVAEPMWREPRQTERGRAYYARFEAWLKENGITEHPDTFASHPPRSLVLIPKALQPHADRVDEDVYTFVGACQGDRAEEGGWQRPAGAEKVVLVSLGSAFTKQPAFYRECVRAFGNLPGWHLVLQIGRKVTPAELGELPDNVEVHDWVPQLAILRQADLFVTHAGAGGSQEGLATATPMIAVPQAVDQFGNADMLQGLGVARKLATEEATADLLRETALALVDDPEVARRLRRIQAEMAQEGGTRRAADLIEAELPARHERQEPVGDRPNGG

SEQ ID NO: 7 - DNA, 894 bp, Artificial Sequence
  Wild type laminaribiose phosphorylase from      Paenibacillus
atgtacccatttggaaaaggctttgcccgttacgttccttttctgctgctgttgattccgctggcgttatacgtcgttttttatttcggaccatcggtgttgaccgtgatttattcgtttacggatgtgaaaaacatccccggcagctccatgaacttcgtcgggctcgataattataaagacttgttttttgccggaaattccggggaacgctgggtttccatcaaaaacacgttaattttcatgtttatcgtcaccatcgtgcagaacggagtggcgttgtttgtggctgtgctgatcaaccaacggctgcgcggcgattatttttaccgggccgtattttttcttccggtcgtgcttggggtggccgttgttgcgctcgtttggggcatgatgttcgatccgatgagcgggccggttaacatgctgtatcaatcgctgttcggatataaagacatgtttttctccagctttacgcatgcgttcggctacattattttcgttcaaatctggatgtacatgggctattcaatgctgatttttctcgccggcctacaatccgtgccaaaagatctgtacgaagcggggtacatcgacggtacgaaccgctggcaatcgttcaagaatattacgttcccgctgatcgccccgtcttttacggtcaatatgctgctttcgatcatcggggcgatgtccacgttcgatattatcctcgccacgacggacggcaagttcaacaccagaacgatggcttacgacgtatataaggaaacgttccgcggcagtctggaaatgggcttgccttcggcgctgtccgtggtccagttcctgatcattttggtgtttgttatcgtggccgtcagacagacgcgtaagagagaggtggaatattaa

SEQ ID NO: 8 - protein, 297 aa, Artificial Sequence
  Wild type laminaribiose phosphorylase from      Paenibacillus
MYPFGKGFARYVPFLLLLIPLALYVVFYFGPSVLTVIYSFTDVKNIPGSSMNFVGLDNYKDLFFAGNSGERWVSIKNTLIFMFIVTIVQNGVALFVAVLINQRLRGDYFYRAVFFLPVVLGVAVVALVWGMMFDPMSGPVNMLYQSLFGYKDMFFSSFTHAFGYIIFVQIWMYMGYSMLIFLAGLQSVPKDLYEAGYIDGTNRWQSFKNITFPLIAPSFTVNMLLSIIGAMSTFDIILATTDGKFNTRTMAYDVYKETFRGSLEMGLPSALSVVQFLIILVFVIVAVRQTRKREVEY

SEQ ID NO: 9 - DNA, 1374 bp, Artificial Sequence
  Variant of UDP-glycosyltransferase 76G1 from      Stevia
atggagaacaaaaccgaaaccaccgtgcgtcgtcgtcgtcgtatcattctgtttccggttccgttccagggccacatcaacccgattctgcaactggcgaacgtgctgtacagcaagggttttagcatcaccatttttcacaccaacttcaacaagccgaaaaccagcaactatccgcacttcacctttcgtttcatcctggacaacgatccgcaggacgagcgtattagcaacctgccgacccatggtccgctggcgggtatgcgtatcccgatcattaacgagcacggcgcggatgaactgcgtcgtgagctggaactgctgatgctggcgagcgaggaagacgaggaagttagctgcctgattaccgatgcgctgtggtacttcgcgcaaagcgtggcggacagcctgaacctgcgtcgtctggttctgatgaccagcagcctgtttaacttccacgcgcacgtgagcctgccgcagtttgacgagctgggctacctggatccggacgataaaacccgtctggaggaacaagcgagcggtttcccgatgctgaaggttaaagatatcaagagcgcgtatagcaactggcagatcctgaaagaaattctgggcaagatgatcaagcagacccgtgcgagcagcggtgtgatttggaacagctttaaagagctggaggaaagcgagctggaaaccgttatccgtgaaattccggcgccgagcttcctgattccgctgccgaagcacctgaccgcgagcagcagcagcctgctggaccacgatcgtaccgtgttccagtggctggaccagcaaccgccgagcagcgtgctgtacgttagctttggcagcaccagcgaggtggacgaaaaggatttcctggagattgcgcgtggtctggttgacagcaaacaaagctttctgtgggtggttcgtccgggcttcgtgaagggtagcacctgggttgagccgctgccggatggttttctgggcgaacgtggtcgtatcgtgaagtgggttccgcaacaagaagtgctggcgcacggcgcgattggtgcgttctggacccacagcggttggaacagcaccctggagagcgtgtgcgaaggcgttccgatgatctttagcgacttcggtctggatcagccgctgaacgcgcgttacatgagcgatgttctgaaagtgggcgtttatctggagaacggctgggagcgtggtgaaatcgcgaacgcgattcgtcgtgtgatggttgacgaggaaggtgagtacatccgtcagaacgcgcgtgttctgaagcaaaaagcggatgttagcctgatgaagggtggcagcagctacgagagcctggaaagcctggttagctatattagcagcctg

SEQ ID NO: 10 - protein, 458 aa, Artificial Sequence
  Variant of UDP-glycosyltransferase 76G1 from      Stevia
MENKTETTVRRRRRIILFPVPFQGHINPILQLANVLYSKGFSITIFHTNFNKPKTSNYPHFTFRFILDNDPQDERISNLPTHGPLAGMRIPIINEHGADELRRELELLMLASEEDEEVSCLITDALWYFAQSVADSLNLRRLVLMTSSLFNFHAHVSLPQFDELGYLDPDDKTRLEEQASGFPMLKVKDIKSAYSNWQILKEILGKMIKQTRASSGVIWNSFKELEESELETVIREIPAPSFLIPLPKHLTASSSSLLDHDRTVFQWLDQQPPSSVLYVSFGSTSEVDEKDFLEIARGLVDSKQSFLWVVRPGFVKGSTWVEPLPDGFLGERGRIVKWVPQQEVLAHGAIGAFWTHSGWNSTLESVCEGVPMIFSDFGLDQPLNARYMSDVLKVGVYLENGWERGEIANAIRRVMVDEEGEYIRQNARVLKQKADVSLMKGGSSYESLESLVSYISSL

SEQ ID NO: 11 - DNA, 1203 bp, Artificial Sequence
  Variant of macrolide glycosyltransferase from      Streptomyces
atgaccagtcgttcccatattgccatgtttagtatcgcggcccatggtcacgtgaacccgtccctggaagtcattcgtgaactggtggcgcgtggtcaccgtgttacctatgccatcccgccggttttcgccggtaaagttgctgaagcgggcgccgaagtgaaactgtggaacagcacgctgccgtctccggatgacgatccgcaggcgtggggtaccacgctgctggataatgttgaaccgtttctggcagacgctattcaggctctgccgcaactgatcgaagcgtatgaaggcgacgaaccggatctggtcctgcatgatatcacctcatatccggctcgtgttctggcccatcgctggggtgtcccggccgtgtcactgtcgccgaatctggttgcctgggaaggctacgaaaaagaagtcgctgaaccgatgtgggcggaaccgaaacgtaccgaacgtggtcgcgcctattacgcacgctttcatgcctggctggaagaaaacggtgttacgctgcatccggatgattttgttggtcgtccggcacgttgcctggtgctgattccgaaagccctgcagccgaatgcagaccgtgtggatgaaggtgtttatagctttgtcggcgcctgtcaaggcgatcgcggtgcagagggtgaatggcgtcgcccggcgggtgcagaaaaagttgccctggtctctctgggcagctcttttaccaaacagccggatttctaccgtgaatgcgtgcgcgccttcggtgaattaccgggctggcatctggtcctgcaaattggtcgccatgtggacccggccgatctgggtgatgtgccggaaaacgtggaagttcgctcatgggttccgcagctggctatcctgaaagaagcggacctgtttgtgacgcatgcaggtgctggcggttcgcaagaaggcctggccaccgcaacgccgatgattgcagtgccgcaggcagttgatcaattcggtaatgcagacatgctgcagggtctgggtgttgcccgtcgcctggataccgaagaagcaaccgctgaagcgctgcgtgcagcggcgctggcgctggttgatgatccggaagttgctcgtcgcgcgcgcgatattcaggcgcagatggcgggtgaaggcggtacccgtcgcgcagcagacctgatcgaagctgaactgccggtgcgtcaggcataa

SEQ ID NO: 12 - protein, 400 aa, Artificial Sequence; identical to SEQ ID NO: 4
  Variant of macrolide glycosyltransferase from      Streptomyces

SEQ ID NO: 13 - DNA, 2733 bp, Artificial Sequence
  Variant of laminaribiose phosphorylase from      Paenibacillus
atgggccagaagggttggaaatttcagggcgaacaaggcgagttccgtctggaacaaccggagcacaacagctacctgtattttccgctggtgaacgaagcgggtatgatgagcgcggttaccccgaacctgcacggcgagatcaccagcggtcacaacaccttcctgatggaaccggtgagcgcggagagcctgcacaacagcaaggcgagccgtaacttttgggtgttcattgaaggttatggtgcgtggagcgttagcggcaacagcgcgcgtcagaacgcggcgcgttttaccggtgaggaagagcgtagcgcggtggaggcgggtttcctgtggcacgcggttacccgtgaaaacgagaaggcgggtctgaaagcgcgtaccgtgagcttcgtgccggttaccgacgataagatcgaactgatgcgtgttaccctgaccaacaccggcaacgcgccgctgaaactgaccccgaccgcggcgattccgctgtatggtcgtagcgcggacgatctgcgtgatcaccgtcacgtgaccagcctgctgcaccgtatctttaccagcgaatacggcattgaggttcagccggcgctgagcttcgacgaacgtggtcaccgtgtgaacaaggttacctatggcgtgtttggtgcggaggcgggtggcaccgcgccggcgggcttctttccggttaccgaagatttcattggcgagggtggcgcgctggactggccggaagcggtggttgcgaaccgtgagccggatgcgcaggcgggcaccgcggtggaaggttatgaggcggttggcgcgctgcgttttgcgccggtggagctggcgccgggcaaaagcgtgagctatgtggttgcgatggttatcagcggcgaccgtattgatgttggtcgttacgcggcggattatctggcggcgggtcgttttgacgcgctgctggagcagaaccgtgcgtactggcgtgacaagctggataccgtgcgtttcagcagcggtgatggcgagcaggacctgtggatgaaatgggttaccctgcaaccgattctgcgtcgtctgtatggtaacagcttcctgccgtaccacgattatggtcgtggtggccgtggctggcgtgatctgtggcaagactgcctggcgctgatggtgatggaaccggcggaagtgcgtcacctgctgctgaacaactacgcgggtgtgcgtatggacggcagcaacgcgaccatcattggtgcgggcccgggtgaatttgtggcggatcgtaacaacatcccgcgtgtttggatggaccatggtgcgtggccgctgatgaccaccctgctgtacctgcaccagagcggtgacctggatctgctgttccagccgcaaagctattttcgtgatgtgttcgttaagcgttgccgtgagcgtgatgcgagctggaccccggagcagggtaacaaactgctgaccgcggacggtcaaatttacgaaggcaccatcctggagcacattctgctgcaaaacatcgtgccgttctttaacgttggcgaacacggtaacattaaactggagggcgcggactggaacgatggtctggacctggcgccggaacgtggtgagagcgttgcgtttaccgcgttctacgcgagcaacctgatggaactgagcgagctgctgctggagctgcagaagcgtaccggcaaagatagcctggacatcgcggaagagatggcgctgctgctggataccctgggcaagccgatcagctatgacagcattcaagaaaaacgtagcctgctggatcgttactatgacgcggtgaccccgcgtgttagcggcaagaaactgctgctggatatccgtaaggtggcggaggatctgaagcgtaaagcggactgggcggttgcgcacctgcgtggtagcgaatggattcagagcaaagagggctacgcgtggtttaacggttactataacaacgacggtgaacgtgtggagggcgatcacccggacggtgttcgtatgaccctgaccggtcaagtgttcgcgatcatgggtggcgttgcgaccgatgaacagaccgagaagattagccaagcggtgaaccgttacctgaaagacgaacgtatcggctatcgtctgaacagccgttttggtggcattcagcaaaacctgggtcgtgcgttcggctttgcgttcggtcacaaggagaacggtgcgatgttcagccacatgaccgtgatgtacgcgaacgcgctgtataaacgtggctttgtgcaggaaggtttcgaggttctggatagcatctaccgtctgagcgcggactttgaaaacagccgtatctacccgggcgttccggaatatattaacgagcgtggccgtggcatgtacacctatctgaccggtagcgcgagctggctgctgctgacccaactgaccgaagtgtatggcgttaagggtcgtttcggcgatctgcgtctggagccgaaactggtgcaggcgcaatttgacggcagcggtgaagcggcggttgagaccctgttcgcgggtcgtatgctgcgtgtggtttatcgtaacccgcaggcggcggagcatggtcaatatcgtgtggatagcgttagcctgaacggtcagagcgttgattgccaaaacgacggcgcgggttgcctgatcggtcgtagcctgattgaagcgctgccggcggatggtgtgcatgagctgatcgttaccctgggtcgtaacattagc

SEQ ID NO: 14 - protein, 911 aa, Artificial Sequence
  Variant of laminaribiose phosphorylase from      Paenibacillus
MGQKGWKFQGEQGEFRLEQPEHNSYLYFPLVNEAGMMSAVTPNLHGEITSGHNTFLMEPVSAESLHNSKASRNFWVFIEGYGAWSVSGNSARQNAARFTGEEERSAVEAGFLWHAVTRENEKAGLKARTVSFVPVTDDKIELMRVTLTNTGNAPLKLTPTAAIPLYGRSADDLRDHRHVTSLLHRIFTSEYGIEVQPALSFDERGHRVNKVTYGVFGAEAGGTAPAGFFPVTEDFIGEGGALDWPEAVVANREPDAQAGTAVEGYEAVGALRFAPVELAPGKSVSYVVAMVISGDRIDVGRYAADYLAAGRFDALLEQNRAYWRDKLDTVRFSSGDGEQDLWMKWVTLQPILRRLYGNSFLPYHDYGRGGRGWRDLWQDCLALMVMEPAEVRHLLLNNYAGVRMDGSNATIIGAGPGEFVADRNNIPRVWMDHGAWPLMTTLLYLHQSGDLDLLFQPQSYFRDVFVKRCRERDASWTPEQGNKLLTADGQIYEGTILEHILLQNIVPFFNVGEHGNIKLEGADWNDGLDLAPERGESVAFTAFYASNLMELSELLLELQKRTGKDSLDIAEEMALLLDTLGKPISYDSIQEKRSLLDRYYDAVTPRVSGKKLLLDIRKVAEDLKRKADWAVAHLRGSEWIQSKEGYAWFNGYYNNDGERVEGDHPDGVRMTLTGQVFAIMGGVATDEQTEKISQAVNRYLKDERIGYRLNSRFGGIQQNLGRAFGFAFGHKENGAMFSHMTVMYANALYKRGFVQEGFEVLDSIYRLSADFENSRIYPGVPEYINERGRGMYTYLTGSASWLLLTQLTEVYGVKGRFGDLRLEPKLVQAQFDGSGEAAVETLFAGRMLRVVYRNPQAAEHGQYRVDSVSLNGQSVDCQNDGAGCLIGRSLIEALPADGVHELIVTLGRNIS

SEQ ID NO: 15 - DNA, 1476 bp, Artificial Sequence
  Sucrose phosphorylase from Leuconostoc
atggaaatccagaacaaagcgatgttcattacctacgcggatagcctgggtaaaaacctgaaggacgttcgtaaagtgctgaaagaggacatcggcgatgcgattggtggcgttcacctgctgccgttctttccgagcaccggtgatcgtggcttcgcgccgagcgactacacccgtgttgatagcgcgtttggtgactggagcgatgtggaagcgctgggcgaggaatactatctgatgttcgactttatgatcaaccacattagccgtgaaagcgttatgtaccaggacttcaagaaaaaccacgatgagagcaagtataaagacttctttatccgttgggagaaattttgggcgaaagcgggtgaaaaccgtccgacccaggcggacgtggatctgatctacaagcgtaaagataaggcgccgacccaagagattaccttcgacgatggtaccaccgaaaacctgtggaacacctttggcgacgagcaaatcgacattgatgttaacagcgcgatcgcgaaagagttcattaagaccaccctggaagatatggtgaaacacggtgcgaacctgatccgtctggacgcgtttgcgtacgcggttaagaaagtggacaccaacgatttctttgttgagccggaaatctgggacaccctgaacgaagtgcgtgaaattctgaccccgctgaaggcggagatcctgccggagattcacgaacactatagcatcccgaagaaaattaacgatcacggttacttcacctatgactttgcgctgccgatgaccaccctgtacaccctgtatagcggcaaaaccaaccagctggcgaaatggctgaagatgagcccgatgaagcaattcaccaccctggacacccacgatggtatcggcgtggttgacgcgcgtgatgtgctgaccgacgaggaaattgattacgcgagcgaggaactgtataaggttggcgcgaacgtgaagaaaacctacagcagcgcgagctataacgacctggatatctaccaaattaacagcacctactatagcgcgctgggtaacgacgatgcggcgtatctgctgagccgtatcttccaggtttttgcgccgggcatcccgcaaatttactatgtgggtctgctggcgggcgaaagcgatatcgagctgctggaaagcagcaaagagggtcgtaacattaaccgtcactactatagcgttgacgaagtgaaagaggaagttaagcgtccggtggttgcgaaactgctgaagctgctgagctggcgtaacaacttcgcggcgtttgacctggatggtagcattgatgtggaaaccccgagcgacaccaccatcaagattacccgtaaagataagagcggcgagaacgtggcggttctggtggcgaacgcggcggacaaaacctttaccatcaccgcgaacggcgaggaaattctggcgaacaccgaggcggacaagcagcaactg

SEQ ID NO: 16 - protein, 492 aa, Artificial Sequence
  Sucrose phosphorylase from Leuconostoc
MEIQNKAMFITYADSLGKNLKDVRKVLKEDIGDAIGGVHLLPFFPSTGDRGFAPSDYTRVDSAFGDWSDVEALGEEYYLMFDFMINHISRESVMYQDFKKNHDESKYKDFFIRWEKFWAKAGENRPTQADVDLIYKRKDKAPTQEITFDDGTTENLWNTFGDEQIDIDVNSAIAKEFIKTTLEDMVKHGANLIRLDAFAYAVKKVDTNDFFVEPEIWDTLNEVREILTPLKAEILPEIHEHYSIPKKINDHGYFTYDFALPMTTLYTLYSGKTNQLAKWLKMSPMKQFTTLDTHDGIGVVDARDVLTDEEIDYASEELYKVGANVKKTYSSASYNDLDIYQINSTYYSALGNDDAAYLLSRIFQVFAPGIPQIYYVGLLAGESDIELLESSKEGRNINRHYYSVDEVKEEVKRPVVAKLLKLLSWRNNFAAFDLDGSIDVETPSDTTIKITRKDKSGENVAVLVANAADKTFTITANGEEILANTEADKQQL

SEQ ID NO: 17 - DNA, 1512 bp, Artificial Sequence
  Sucrose phosphorylase from Bifidobacterium
atgaagaacaaagttcaactgatcacctacgcggaccgtctgggtgatggcaccatcaagagcatgaccgatattctgcgtacccgtttcgacggtgtttatgatggcgtgcacatcctgccgttctttaccccgttcgacggtgcggatgcgggctttgacccgattgatcacaccaaggttgacgagcgtctgggtagctgggacgatgtggcggaactgagcaaaacccacaacatcatggttgatgcgattgtgaaccacatgagctgggagagcaagcagtttcaagacgttctggcgaaaggcgaggaaagcgaatactatccgatgttcctgaccatgagcagcgtgtttccgaacggtgcgaccgaggaagacctggcgggtatctatcgtccgcgtccgggtctgccgttcacccactataagtttgcgggtaaaacccgtctggtgtgggttagcttcaccccgcagcaagttgatattgacaccgatagcgacaagggctgggagtacctgatgagcatctttgatcaaatggcggcgagccacgttagctacattcgtctggacgcggtgggttatggcgcgaaagaagcgggtaccagctgcttcatgaccccgaagacctttaaactgatcagccgtctgcgtgaggaaggtgtgaagcgtggcctggagatcctgattgaagttcacagctactataagaaacaggtggagattgcgagcaaagtggaccgtgtttatgattttgcgctgccgccgctgctgctgcatgcgctgagcaccggtcatgtggaaccggttgcgcactggaccgatatccgtccgaacaacgcggtgaccgttctggacacccacgatggtattggcgttatcgacattggtagcgaccaactggatcgtagcctgaagggtctggtgccggatgaggacgttgataacctggtgaacaccatccacgcgaacacccacggtgaaagccaagcggcgaccggcgcggcggcgagcaacctggatctgtaccaagttaacagcacctactatagcgcgctgggttgcaacgaccagcactatattgcggcgcgtgcggtgcagttctttctgccgggcgtgccgcaagtttactatgtgggtgcgctggcgggcaagaacgacatggagctgctgcgtaaaaccaacaacggtcgtgatatcaaccgtcactactatagcaccgcggagattgacgaaaacctgaagcgtccggtggttaaagcgctgaacgcgctggcgaagttccgtaacgaactggacgcgttcgatggtacctttagctacaccaccgacgatgacaccagcatcagcttcacctggcgtggcgagaccagccaggcgaccctgacctttgaaccgaaacgtggtctgggcgttgacaacaccaccccggtggcgatgctggagtgggaagacagcgcgggtgatcaccgtagcgatgacctgattgcgaacccgccggtggttgcg

SEQ ID NO: 18 - protein, 504 aa, Artificial Sequence [sequence omitted]

SEQ ID NO: 19 - DNA, 1479 bp, Artificial Sequence
  Sucrose phosphorylase from uncultured bacterium
atgaagaacaaagtgcagctgattgcgtacgttgaccgtatcagcggtggcggtttccgtaagctgcatgcgctgctgaccggtccgctggcggagatttttggcggtgcgcacctgctgccgttctttaccccgattgatggtgcggatgcgggttttgatccgagcgatcacacccaagtggacccgcgtctgggtacctgggacgatgttcgtatcctgggcggtgcgattgaactggtggcggatctgattgtgaaccacgttagcagcagcagcccgcaatttatcgactacagcaagaaaggcagcgatagcctgtacgcgggcatgttcctgacctatgaccgtgtttttccggagggtgcgcgtgaagcggatatcctgcgtatttatcgtccgcgtccgaccctgccgtttagcccggtgaccctgagcagccgtgagcgtaaactgctgtggaccacctttaacccggaacaggtggacattgatgttcgtcacccggaggcggaagcgtacctgcacagcatcctgaagaaattccaagcggcgggcatccgtatgattcgtctggacgcggttggctatgcgatcaagaaaccgggtgcgagctgctttatgattccggagaccttcgattttatcgcggagctgaccgaaaaggcgcgtgcgctgggtattgaggtgctggttgaaatccacagccactaccgtaaacagatcgaaattgcgcgtcaagtggactgggtttatgattttgcgctgccgccgctggtgctgcatgcgctgtttgcgagcgacccgcacccgctggcgcagtggctgagcatcagcccgcgtaacgcggtgaccgttctggacacccacgatggcatcggtgtgattgatgttggtgcggatgcggaaggtaacccgggtctgctgagcccggcggcgattgacagcctggttgagaccatccacagccgtagccaaggtcaaagccgtgaagcgaccggtgcggcggcgaacaacctggatctgtaccaagtgaactgcaccttcctggatgcgctgggcggtcgtgaaccggattatctgattgcgcgtgcgctgcagttctttgcgccgggcatcccgcaagtgtactatgttggtctgctgggcggtaccaacgacatggatctgctgggtcgtagcggtgtgggtcgtgacattaaccgtcactactataccgacgcggagattgatgcggcgctggcgcgtccgctggtgcgtaccctgatcgcgctgattcgtctgcgtaacacccacccggcgttcgcgggtgaatttgatgtgagcgttccggcggcgacccagattcgtctgcgttggcagcgtcaagagcactggatcgaactgcacgttgacctgagcatcccgaaggcgagcattaccggcaccggtatccacccgatcaccattccgggtgcggcggatgcgggtgcgccgagc

SEQ ID NO: 20 - protein, 493 aa, Artificial Sequence
  Sucrose phosphorylase from uncultured bacterium
MKNKVQLIAYVDRISGGGFRKLHALLTGPLAEIFGGAHLLPFFTPIDGADAGFDPSDHTQVDPRLGTWDDVRILGGAIELVADLIVNHVSSSSPQFIDYSKKGSDSLYAGMFLTYDRVFPEGAREADILRIYRPRPTLPFSPVTLSSRERKLLWTTFNPEQVDIDVRHPEAEAYLHSILKKFQAAGIRMIRLDAVGYAIKKPGASCFMIPETFDFIAELTEKARALGIEVLVEIHSHYRKQIEIARQVDWVYDFALPPLVLHALFASDPHPLAQWLSISPRNAVTVLDTHDGIGVIDVGADAEGNPGLLSPAAIDSLVETIHSRSQGQSREATGAAANNLDLYQVNCTFLDALGGREPDYLIARALQFFAPGIPQVYYVGLLGGTNDMDLLGRSGVGRDINRHYYTDAEIDAALARPLVRTLIALIRLRNTHPAFAGEFDVSVPAATQIRLRWQRQEHWIELHVDLSIPKASITGTGIHPITIPGAADAGAPS

SEQ ID NO: 21 - DNA, 1467 bp, Artificial Sequence
  Sucrose phosphorylase from Oenococcus
atgccggtgaagaacaaagcgatgctgatcacctacagcgatagcatgggtaaaaacatcaaggaactgcagtacattctggacaaatatatcggcgatgcgattggtggcgttcacctgctgccgttctttccgagcaccggtgatcgtggcttcgcgccgagcgactatacccgtgtgaacccggactttggtgattgggaggacgttgaggaactgggcaagaaatactatctgatgttcgactttatgatcaaccacattagccgtgaaagcattatgtaccaggacttcaaagagaagaaagatgcgagcagctataaggacttctttatccgttgggaaaaattttggccgaagggtcgtccgaccaaggcggacatcgatctgatttacaagcgtaaagataaggcgccgatccagggtattaccttcgcggacggcagccaagaacacctgtggaacacctttggtgacgagcaaatcgatattaacgtgaaaagcaaggttgcgcaggagttctttaaagataccctgcaaagcatggtgaagcacggcgcggacctgattcgtctggatgcgttcgcgtacgcgatcaagaaaattgataccaacgacttctttatcgaaccggagatttgggatctgctggaaagcgttcgtaaaattctggacccgctgcatgcggagatcctgccggaaatttacgagcactataccatcccggcgaagattaacgaatacggttatttcacctacgactttgtgctgccgctggttatcctgtacaccctgtatagcggcaacccgaaacagctggcgaaatggctgaagatgagcccgaagaaacaattcaccaccctggatacccacgacggtatcggcgtggttgatgcgcgtgacatcctgaccgacgaggaaattgattacaccagcagcgagctgtataaagtgggtgcgaacgttaagcgtacctacagcagcgcggcgtataacaacctggacatctaccagattaacagcacctactatagcgcgctgggtaacgacgataaagcgtatctgctggcgcgtgcgatccagatttttgcgccgggcatcccgcaaatttactatgcgggtctgctggcgggcgaaaacgacctggatctgctggaaaaaaccaaagagggtcgtaacattaaccgtcactactatagcgaggaagaggtggcgaacgaggttcaacgtccgatcgttgcgtgcctgctgaagctgctggcgtggcgtaaccgtagcgcggcgttcgacctgcagggtgatattcaagttagcgcgaccgataaaaacgaaatcaagatcattcgtaccagcaccaacggtcaagacaccgcggagctgaccgcgaacgtggcgctgaaaacctttaccatcaaagaaaacgacaagatcattctgattgaggatcaaaccgacaccaaggatatc

SEQ ID NO: 22 - protein, 489 aa, Artificial Sequence
  Sucrose phosphorylase from Oenococcus
MPVKNKAMLITYSDSMGKNIKELQYILDKYIGDAIGGVHLLPFFPSTGDRGFAPSDYTRVNPDFGDWEDVEELGKKYYLMFDFMINHISRESIMYQDFKEKKDASSYKDFFIRWEKFWPKGRPTKADIDLIYKRKDKAPIQGITFADGSQEHLWNTFGDEQIDINVKSKVAQEFFKDTLQSMVKHGADLIRLDAFAYAIKKIDTNDFFIEPEIWDLLESVRKILDPLHAEILPEIYEHYTIPAKINEYGYFTYDFVLPLVILYTLYSGNPKQLAKWLKMSPKKQFTTLDTHDGIGVVDARDILTDEEIDYTSSELYKVGANVKRTYSSAAYNNLDIYQINSTYYSALGNDDKAYLLARAIQIFAPGIPQIYYAGLLAGENDLDLLEKTKEGRNINRHYYSEEEVANEVQRPIVACLLKLLAWRNRSAAFDLQGDIQVSATDKNEIKIIRTSTNGQDTAELTANVALKTFTIKENDKIILIEDQTDTKDI

SEQ ID NO: 23 - DNA, 1446 bp, Artificial Sequence
  Sucrose phosphorylase from Enterococcus
atgaagattaaaaacgaggcgatgctgatcacctactgcgacagcctgggtaacaacctgaaagacctgaaccaggtgctggatcagcacctgcaaggcgtggttggtggcatccacctgctgccgttctttccgagcaccggtgatcgtggcttcgcgccgagcgactataccgtggttgatagcgcgtttggtacctggcaagaggttgaagcgctgggcgagaagtactacctgatgttcgacttcatgatcaaccacatcagccgtgaaagcaaattctttcaggatttcaagcaaaaccacgaggcgagcccgtacaaggaaatgtttattcgtatccacgagttctttccggaaaaccgtccgaccccggaggacattgatctgatctataagcgtaaagacaaggcgccgttccaggaagtgacctttgcggatggtaccaccgaacaggtttggaacaccttcggcgaggaacaaattgacctggatgtgaacaaagaagttaccaagcaattcatcaaggacaccctgaaggatatggcgagccacggttgcagcctgattcgtctggacgcgttcgcgtacgcgatcaagaaactggacaccaacgatttctttgtggagccggaaatttgggacctgctggatgaggttcgtgaggaagcggcgaaatacaactgcgaactgctgccggagatccacgaacactataccattcagatgaagatcgcggaccacgattactatgtgtacgacttcgcgctgccgatgatcaccctgtacagcctgtatagcggtaaaaccaaccgtctggcgaactggctgaaaatgagcccgatgaagcaatttaccaccctggacacccacgatggtattggcgtggttgacgcgaaagatctgctgaccgacgaggaactggattacaccagcgaggaactgtataaagtgggcgcgaacgttaagaaaatctacagcagcgcgagctataacaacctggacatttaccagatcaacagcacctactatagcgcgctgggtgacaacgatcgtagctatctgctggcgcgtgcgattcagtgctttgcgccgggcattccgcaaatctactatgtgggtctgctggcgggcaaaaacgacctggagctgctggaaagcaccaaagagggtcgtaacattaaccgtcactactatagcctgaccgaaatcgatgaggaagttaaccgtccggtggttaaggcgctgttcgacctgctgcgttttcgtaaccgtgcggcggcgtttgatctggcgggcgagatcaccgtggaaaccccgagcgaggaaaccatcattatcacccgtaaagacgcgaacggtgatcgtgcggttctgaccgcgaacctgcagaccaaggactttaccgtgacccacaacggcgaactggttctgcagcaactggat

SEQ ID NO: 24 - protein, 482 aa, Artificial Sequence
  Sucrose phosphorylase from Enterococcus
MKIKNEAMLITYCDSLGNNLKDLNQVLDQHLQGVVGGIHLLPFFPSTGDRGFAPSDYTVVDSAFGTWQEVEALGEKYYLMFDFMINHISRESKFFQDFKQNHEASPYKEMFIRIHEFFPENRPTPEDIDLIYKRKDKAPFQEVTFADGTTEQVWNTFGEEQIDLDVNKEVTKQFIKDTLKDMASHGCSLIRLDAFAYAIKKLDTNDFFVEPEIWDLLDEVREEAAKYNCELLPEIHEHYTIQMKIADHDYYVYDFALPMITLYSLYSGKTNRLANWLKMSPMKQFTTLDTHDGIGVVDAKDLLTDEELDYTSEELYKVGANVKKIYSSASYNNLDIYQINSTYYSALGDNDRSYLLARAIQCFAPGIPQIYYVGLLAGKNDLELLESTKEGRNINRHYYSLTEIDEEVNRPVVKALFDLLRFRNRAAAFDLAGEITVETPSEETIIITRKDANGDRAVLTANLQTKDFTVTHNGELVLQQLD

SEQ ID NO: 25 - DNA, 1440 bp, Artificial Sequence
  Sucrose phosphorylase from Streptococcus
atgagcatccagaacaagaccatgctgattacctacagcgatagcctgggtaacaacctgaaggacctgtacgagaacctggaaaaatatttcggcgatgcggtgggtggcgttcacctgctgccgttctttccgagcaccggtgatcgtggcttcgcgccggtggattatgaccaggttgacccggcgtttggtgattgggaggacgtgaagcgtctgggcgataagtactacctgatgttcgacttcatgatcaaccacattagccgtcaaagcaagtactataaaaactaccaggagaagcacgaccaaagcgcgtataaagacctgttcctgaactgggacaagttttggccggaaaaccgtccgacccaggcggatgttgacctgatctacaagcgtaaagatcgtgcgccgaaacaagagattaccttcgcggacggtaccaccgaacacctgtggaacacctttggcgaggaacaaatcgatctggacgtgaccaaagaggtgaccatggacttcatccgtaaaaccattgaacacctggttagcaacggttgcgacctgattcgtctggatgcgtttgcgtacgcggtgaagaaactggataccaacgacttctttgttgagccggatatctgggacctgctggataaggttcgtgatatggcggcgagctacggcgcggaactgctgccggagattcacgaacactatagcatccagttcaaaattgcggatcacgactactatgtgtacgactttgcgctgccgatggttaccctgtacaccctgtatagcagcaaggtggagcgtctggcgaagtggctgaaaatgagcccgatgaaacaattcaccaccctggacacccacgatggtatcggcgtggttgacgttaaggatatcctgaccgacgaggaaattgattacgcgagcaacgagctgtataaagtgggtgcgaacgttaagcgtaaatacagcaccgcggaatataacaacctggatatctaccagattaacagcacctactatagcgcgctgggtgacgatgacagcaagtatttcctggcgcgtctgatccaggcgtttgcgccgggcattccgcaagtgtactatgttggttttctggcgggcaagaacgacctggagctgctggaaaacaccaaagaaggtcgtaacatcaaccgtcactactatagcaacgaggaaattgcgcaggaagtggaacgtccggtggttaagagcctgctgaaactgttcagctttcgtaacaacagccaagcgttcgatctggagggcagcatcgaggtggaaaccccggacgaacacaccatcgttattacccgtcagaacaaagaccaaaccgcgaccgcggtggcgcgtatcaacctggcggatggtacctaccaggttaccgagaacggccaagacattgaattt

SEQ ID NO: 26 - protein, 480 aa, Artificial Sequence
  Sucrose phosphorylase from Streptococcus
MSIQNKTMLITYSDSLGNNLKDLYENLEKYFGDAVGGVHLLPFFPSTGDRGFAPVDYDQVDPAFGDWEDVKRLGDKYYLMFDFMINHISRQSKYYKNYQEKHDQSAYKDLFLNWDKFWPENRPTQADVDLIYKRKDRAPKQEITFADGTTEHLWNTFGEEQIDLDVTKEVTMDFIRKTIEHLVSNGCDLIRLDAFAYAVKKLDTNDFFVEPDIWDLLDKVRDMAASYGAELLPEIHEHYSIQFKIADHDYYVYDFALPMVTLYTLYSSKVERLAKWLKMSPMKQFTTLDTHDGIGVVDVKDILTDEEIDYASNELYKVGANVKRKYSTAEYNNLDIYQINSTYYSALGDDDSKYFLARLIQAFAPGIPQVYYVGFLAGKNDLELLENTKEGRNINRHYYSNEEIAQEVERPVVKSLLKLFSFRNNSQAFDLEGSIEVETPDEHTIVITRQNKDQTATAVARINLADGTYQVTENGQDIEF

SEQ ID NO: 27 - DNA, 1509 bp, Artificial Sequence
  Sucrose phosphorylase from Scardovia
atgaagaacaaagttcagctgattacctacgcggatcgtctgggtgaaggtaccctggcgagcctgaccgatgtgctgcgtacccgtttcgatggcgtttatgaaggtgtgcacattctgccgttctttaccccgttcgacggcgcggatgcgggttttgacccgatcgatcacaccaaggttgacccgcgtctgggtagctgggacgatgtggcggagctgagcaaaacccacgacatcatggttgatgcgattgtgaaccacatgagctggcaaagcgaacagtttcaagatgttatgaagaaaggtgaaggcagcgaatactatccgatgttcctgaccatgagcgcggtgtttccggagggtgcgaccgaggaagacctggcgggtatttatcgtccgcgtccgggtctgccgttcacccactatacctggggtggcaagacccgtctggtttggaccacctttaccccgcagcaagtggacatcgataccgcgagcgacgaaggttgggcgtacctgatgagcatcctggatcagatgagcaaaagccacgttagcttcattcgtctggacgcggtgggttatggcgcgaagcgtgcgaaaaccagctgcttcatgaccccggacacctttgatctgatcagccgtattaaagaggagagcgcgcgtcgtggcatggagaccctgatcgaagttcacagctactataagaaacaagtggagattgcgaacaaggtggaccgtgtttacgattttgcgatcccggcgctgctgctgcatgcgctgagcaccggtaaaaccggtccgctggcgcactggctgggtgtgcgtccggttaacgcggtgaacgttctggacacccacgatggtatcggcgtgattgatgttggcagcgaccagatggatcgtagcctgaagggtctggttccggacgatgaagttgaccaactggtggagaccattaaagaaaacagccatggtgaaagcgcggaagcgaccggtgcggcggcgagcaacctggatctgtaccaggttaactgcacctactatagcgcgctgggctgcgacgatcaaaaatacatcgcgagccgtgcggtgcagttctatatgccgggtgtgccgcaagtttactatgtgggcgcgctggcgggcaagaacgacatggagctgctgaaacgtaccaacgttggtcgtgatatcaaccgtcactactataccgtggcggagattgacgaaaacctgcgtcgtccggtggttcaggcgctgaacgcgctgggccgtttccgtaacaccctggaggcgtttgacggtgatttcagctttaccgaaaacggtggcagcctggcgatgacctggaccggtcaaagcaccagcaccaccctgacctttgcgccgggcaagggtgcgggtagcagcggcgtgccggtttgcaccatcgcgtggaaagacgcgagcggtgaacacgtgaccgacgatatcattgcgaacccgccggcggcggcgcag

SEQ ID NO: 28 - protein, 503 aa, Artificial Sequence
  Sucrose phosphorylase from Scardovia
MKNKVQLITYADRLGEGTLASLTDVLRTRFDGVYEGVHILPFFTPFDGADAGFDPIDHTKVDPRLGSWDDVAELSKTHDIMVDAIVNHMSWQSEQFQDVMKKGEGSEYYPMFLTMSAVFPEGATEEDLAGIYRPRPGLPFTHYTWGGKTRLVWTTFTPQQVDIDTASDEGWAYLMSILDQMSKSHVSFIRLDAVGYGAKRAKTSCFMTPDTFDLISRIKEESARRGMETLIEVHSYYKKQVEIANKVDRVYDFAIPALLLHALSTGKTGPLAHWLGVRPVNAVNVLDTHDGIGVIDVGSDQMDRSLKGLVPDDEVDQLVETIKENSHGESAEATGAAASNLDLYQVNCTYYSALGCDDQKYIASRAVQFYMPGVPQVYYVGALAGKNDMELLKRTNVGRDINRHYYTVAEIDENLRRPVVQALNALGRFRNTLEAFDGDFSFTENGGSLAMTWTGQSTSTTLTFAPGKGAGSSGVPVCTIAWKDASGEHVTDDIIANPPAAAQ

SEQ ID NO: 29 - DNA, 1506 bp, Artificial Sequence
  Sucrose phosphorylase from Parascardovia
atgaagaacaaagttcagctgatcacctacgcgaaccgtttcggcgagggcaccattcgtagcctgaccgacgtgctgcgtacccgttttgatggtgtttacgaaggcgtgcacattctgccgttctttaccccgttcgacggtgcggatgcgggctttgacccgattgatcacaccaaggttgatccgcgtctgggtacctgggacgatatcgcggagctgagcaaaacccacgacatcatggttgatgcgattgtgaaccacatgagctgggagagcaagcagttccaagacgttatgaaaaacggtgaagcgagcccgtactatggcatgttcctgaccatgagcgcggtgtttccggatggtgcgaccgaggaagatctggcgggtatctatcgtccgcgtccgggtctgccgttcacccactataactggggtggcaagacccgtctggtttggaccacctttaccccgcagcaagtggacatcaacaccgatagcattgaaggctgggactacctgatgagcatcctggatcagatgagccaaagccacgttagcttcattcgtctggacgcggtgggttatggcgcgaagcaggcgaaaaccacctgcttcatgaccccgaagacctttgagctgatcggtcgtattaaagaggagagcgcgaaacgtggcatggagaccctgatcgaagttcacagccactacatgaagcaagtggaaattgcgggtaaagtggaccgtgtttatgattttgcgattccgggtctgctgctgcatgcgctgaccaccggtaaaaccggtccgctggcgcgttggatggaagtgcgtccgggtaacgcggtgaacgttctggacacccacgatggtattggcgttatcgatattggtagcgaccagatggatcgtagcctgaagggtctggtgccggacgaggaagttgatcaactggtggagaccatcaaagaaaacagccacggtgaaagcgcggcggcgaccggcgcggcggcgagcaacctggacctgtaccaggttaactgcacctactatagcgcgctgggttgcgacgatcaaaaatatattgcggcgcgtgcggtgcagttctttatgccgggcgtgccgcaagtttactatgtgggtgcgctggcgggcgagaacgacatgcagctgctgaaggaaaccaacgttggtcgtgatatcaaccgtcactactatagcctggaggaaattgaccaaaacctggagcgtccggtggttaaagcgctgaacgcgctgtgccgtttccgtaacggtctggaagcgtttgacggcgatttcagctttgcgcagaacggtgatagcctgaccttcgcgtggaccggcaaggagaccgaagcgaccctgacctttaccccggcgcaaggtgttggcaaagatgaggcgccggtgtgcagcctgcgttggaaggacagcgcgggtgatcacaaaaccgatgatctgattgcggaaccgccggcggtggcg

SEQ ID NO: 30 - protein, 502 aa, Artificial Sequence
  Sucrose phosphorylase from Parascardovia
MKNKVQLITYANRFGEGTIRSLTDVLRTRFDGVYEGVHILPFFTPFDGADAGFDPIDHTKVDPRLGTWDDIAELSKTHDIMVDAIVNHMSWESKQFQDVMKNGEASPYYGMFLTMSAVFPDGATEEDLAGIYRPRPGLPFTHYNWGGKTRLVWTTFTPQQVDINTDSIEGWDYLMSILDQMSQSHVSFIRLDAVGYGAKQAKTTCFMTPKTFELIGRIKEESAKRGMETLIEVHSHYMKQVEIAGKVDRVYDFAIPGLLLHALTTGKTGPLARWMEVRPGNAVNVLDTHDGIGVIDIGSDQMDRSLKGLVPDEEVDQLVETIKENSHGESAAATGAAASNLDLYQVNCTYYSALGCDDQKYIAARAVQFFMPGVPQVYYVGALAGENDMQLLKETNVGRDINRHYYSLEEIDQNLERPVVKALNALCRFRNGLEAFDGDFSFAQNGDSLTFAWTGKETEATLTFTPAQGVGKDEAPVCSLRWKDSAGDHKTDDLIAEPPAVA

SEQ ID NO: 31 - DNA, 1500 bp, Artificial Sequence
  Sucrose phosphorylase from Alloscardovia
atgaagaacaaagttcaactgattacctacgcggatcgtctgggtgacggcaccctgaagagcatgaccgagaccctgcgtaaacacttcgagggtgtttatgaaggcgtgcacatcctgccgttctttaccccgttcgatggtgcggacgcgggctttgatccggttgaccacaccaaagtggacccgcgtctgggtagctgggacgatgtggcggaactgagcaccacccacgatatcatggttgacaccattgtgaaccacatgagctgggagagcgaacagttccaagatgttatggcgaagggcgaggacagcgaatactatccgatgttcctgaccatgagcagcatttttccggatggtgtgaccgaggaagacctgaccgcgatctatcgtccgcgtccgggtctgccgttcacccactataactggggtggcaaaacccgtctggtttggaccacctttaccccgcagcaagtggacattgataccgacagcgagatgggttggaactacctgctgagcatcctggatcagctgagccaaagccacgttagccaaattcgtctggacgcggtgggttatggcgcgaaggagaaaaacagcagctgcttcatgaccccgaagacctttaaactgatcgaacgtattaaggcggagggtgaaaaacgtggcctggagaccctgatcgaagttcacagctactataagaaacaggtggagattgcgagcaaggtggatcgtgtttacgactttgcgatcccgggtctgctgctgcatgcgctggaatttggcaaaaccgatgcgctggcgcaatggattgacgttcgtccgaacaacgcggtgaacgttctggatacccacgacggtatcggcgttatcgatattggtagcgatcagatggaccgtagcctggcgggtctggtgccggatgaggaagttgacgcgctggttgagagcatccaccgtaacagcaagggtgaaagccaggaagcgaccggcgcggcggcgagcaacctggacctgtaccaagttaactgcacctactatgcggcgctgggtagcgacgatcagaaatatattgcggcgcgtgcggtgcagttctttatgccgggcgtgccgcaagtttactatgtgggtgcgctggcgggcagcaacgatatggacctgctgaagcgtaccaacgtgggtcgtgatatcaaccgtcactactatagcgcggcggaagtggcgagcgaggtggaacgtccggtggttcaggcgctgaacgcgctgggccgtttccgtaacaccctgagcgcgttcgatggtgaatttagctacagcaacgcggacggcgttctgaccatgacctgggcggatgatgcgacccgtgcgaccctgacctttgcgccgaaggcgaacagcaacggtgcgagcgtggcgcgtctggagtggaccgatgcggcgggtgaacatgcgaccgacgatctgatcgcgaacccgccggtggttgcg

SEQ ID NO: 32 - protein, 500 aa, Artificial Sequence
  Sucrose phosphorylase from Alloscardovia
MKNKVQLITYADRLGDGTLKSMTETLRKHFEGVYEGVHILPFFTPFDGADAGFDPVDHTKVDPRLGSWDDVAELSTTHDIMVDTIVNHMSWESEQFQDVMAKGEDSEYYPMFLTMSSIFPDGVTEEDLTAIYRPRPGLPFTHYNWGGKTRLVWTTFTPQQVDIDTDSEMGWNYLLSILDQLSQSHVSQIRLDAVGYGAKEKNSSCFMTPKTFKLIERIKAEGEKRGLETLIEVHSYYKKQVEIASKVDRVYDFAIPGLLLHALEFGKTDALAQWIDVRPNNAVNVLDTHDGIGVIDIGSDQMDRSLAGLVPDEEVDALVESIHRNSKGESQEATGAAASNLDLYQVNCTYYAALGSDDQKYIAARAVQFFMPGVPQVYYVGALAGSNDMDLLKRTNVGRDINRHYYSAAEVASEVERPVVQALNALGRFRNTLSAFDGEFSYSNADGVLTMTWADDATRATLTFAPKANSNGASVARLEWTDAAGEHATDDLIANPPVVA